(19) United States
(12) Patent Application Publication
(10) Pub. No.: US 2004/0229224 A1
(43) Pub. Date: Nov. 18, 2004

(54) Allele-specific expression patterns

(75) Inventors: Kelly A. Frazer, San Mateo, CA (US); David R. Cox, Belmont, CA (US); Heng Tao, Mountain View, CA (US); Krishna Pant, Milpitas, CA (US); Geoffrey Nilsen, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Perlegen Sciences, Inc., Mountain View, CA

(21) Appl. No.: 10/438,184

(22) Filed: May 13, 2003

(51) Int. Cl. C12Q 1/68

(52) U.S. Cl. 435/6

(57) ABSTRACT

The invention provides methods of analyzing genes for differential relative allelic expression patterns. Haplotype blocks throughout the genomes of individuals are analyzed to identify haplotype patterns that are associated with specific differential relative allelic expression patterns. Haplotype blocks that contain associated haplotype patterns may be further investigated to identify genes or variants of genes involved in differential relative allelic expression patterns.

### BACKGROUND OF THE INVENTION

The DNA that makes up human chromosomes provides the instructions that direct the production of all proteins in the body. These proteins carry out the vital functions of life. Variations in DNA often produce variations in the proteins, thus affecting the function of cells. Although environment often plays a significant role, variations or mutations in DNA are directly related to almost all human diseases, including infectious diseases, cancer, inherited disorders, and autoimmune disorders. Moreover, knowledge of human genetics has led to the realization that many diseases result from either complex interactions of several genes or from any number of mutations within one gene. For example, Type I and II diabetes have been linked to multiple genes, each with its own pattern of mutations. In contrast, cystic fibrosis can be caused by any one of over 300 different mutations in a single gene.

The correlation of genotypes with phenotypes has in the past been performed using different strategies. One strategy is the candidate gene approach, in which a gene that has a known function is analyzed in patients who have a disease in which the gene is thought to play a role. For example, if the phenotype is hypertension, genes that are known to play a role in the regulation of blood pressure are analyzed. This approach is limited in utility because it only provides for the investigation of genes with known functions. It is estimated that of the approximately 40,000 genes in the human genome, less than half of those genes currently have known or predicted functions (Lander et al., Nature 2001 Feb. 15;409(6822):860-921). Although variant sequences of candidate genes may be identified using this approach, it is inherently limited by the fact that variant sequences in other genes that contribute to the phenotype will be necessarily missed when the technique is employed.

Another strategy involves whole-genome analysis using variable number tandem repeat (VNTR) markers. It is well known that short stretches of DNA in the genome of mammalian species are repeated any number of times, such as (GAC)N in which n is usually any number ranging from 5 to 100. These sequences are analyzed in the genome of patients who have a particular phenotype to determine if a particular length of repeat at a given locus in the genome correlates with the phenotype. This approach is limited in that the markers are not spread evenly throughout the genome and the presence of a particular length of repeated sequences is not necessarily indicative or predictive of any other variant sequences located near the marker.

Because any two humans are 99.9% similar in their genetic makeup, most of the sequence of the DNA of their genomes is identical. However, there are variations in DNA sequence between individuals. For example, there are deletions of many-base stretches of DNA, insertion of stretches of DNA, variations in the number of repetitive DNA elements in noncoding regions, and changes in single nitrogenous base positions in the genome called single nucleotide polymorphisms or “SNPs.”

The candidate gene and VNTR methods of discovering genotypes that correlate with phenotypes such as disease states are useful in determining the genetic causes of rare diseases, and both methods have been used successfully for this purpose. Unlike rare diseases and other rare phenotypes, common diseases and other common phenotypes are frequently caused by multiple genetic variants that occur in disparate locations throughout the genome. Candidate gene methods, which only analyze genes of known function, and VNTR methods, which rely on widely spaced markers, are of limited utility in elucidating genotypes that are associated with common phenotypes.

### BRIEF SUMMARY OF THE INVENTION

The invention provides methods of characterizing a gene. The methods involve determining a differential relative allelic expression pattern of at least two alleles of the gene from samples containing diploid cells from a plurality of individuals of the same species, wherein the cells are heterozygous for the gene. One then determines whether the differential relative allelic expression pattern of the gene is associated with the presence of a haplotype pattern of one or more polymorphic forms at polymorphic sites in a haplotype block. In such methods, if the haplotype block has only a single polymorphic site, the polymorphic site is outside the transcribed region of the gene and regulatory regions that control the transcription thereof.

In some methods, the haplotype pattern of polymorphic forms is determined by detecting a polymorphic form at a haplotype-defining polymorphic site within the haplotype block. In some methods, the haplotype pattern of polymorphic forms is determined by detecting a plurality of polymorphic forms at a plurality of polymorphic sites within the haplotype block. In some methods, the polymorphic sites are SNPs. In some methods, the individuals are humans. In some methods, the differential relative allelic expression pattern is determined from a plurality of diploid cells obtained directly from a mammalian organism. In some methods, the diploid cells are cultured before step (a) is performed. In some methods, the haplotype block comprises at least ten polymorphic sites. In some methods, the haplotype block comprises between one and ten polymorphic sites. In some methods, the haplotype block comprises only one polymorphic site. In some methods, the haplotype block is on a different chromosome than the gene. In some methods, the haplotype block is on the same chromosome as the gene. In some methods, all polymorphic sites in the haplotype block are located at least 10 kb away from the gene. In some methods, at least one of the polymorphic sites in the haplotype block is not located within promoter, enhancer, or intronic sequences of the gene. In some methods, at least one polymorphic site of the haplotype block is within the gene. In some methods, the haplotype block is at least 50 kb distant from the gene. In some methods, the haplotype block spans at least 10 kb. In some methods, at least 80% of the haplotype patterns of one or more polymorphic sites in the haplotype block in the population are one of four or fewer distinct haplotype patterns.

In some methods, one determines which of the haplotype patterns at each of a plurality of haplotype blocks are associated with the differential relative allelic expression pattern. In some methods, one haplotype block is within 50 kb of the gene, and a second haplotype block is at least 100 kb away from the gene on the same chromosome or is located on a different chromosome. In some methods, the haplotype block is within 50 kb of the gene, and a first haplotype pattern of the haplotype block is associated with the differential relative allelic expression pattern, and the method further comprises repeating step (b) with a second haplotype block at least 100 kb from the gene or located on a different chromosome in a subset of the samples from individuals having the first haplotype pattern that is associated with the differential relative allelic expression pattern.

In some methods, the plurality of haplotype blocks comprises at least 25,000 blocks of polymorphic sites. In some methods, the plurality of haplotype blocks comprises at least 100,000 blocks of polymorphic sites. In some methods, the plurality of haplotype blocks comprises at least 200,000 blocks of polymorphic sites. In some methods, the plurality of haplotype blocks comprises at least 500,000 blocks of polymorphic sites. In some methods, the plurality of haplotype blocks comprises at least 1,000,000 blocks of polymorphic sites. In some methods, substantially all regions of the genome of the individuals are analyzed for association of haplotype patterns to the differential relative allelic expression pattern.

Some methods further comprise performing a clinical trial in which the identity of a drug a patient receives is determined by presence or absence in the patient of a haplotype pattern that is associated with the differential relative allelic expression pattern. Some methods further comprising performing a clinical trial in which the dose of a drug a patient receives is determined by presence or absence in the patient of a haplotype pattern that is associated with the differential relative allelic expression pattern. Some methods further comprise performing a clinical trial in which the dose and identity of a drug a patient receives is determined by presence or absence in the patient of a haplotype pattern that is associated with the differential relative allelic expression pattern. Some methods further comprise performing a clinical trial in which a haplotype pattern that is associated with the differential relative allelic expression pattern is further analyzed to determine if the haplotype pattern is also associated with efficacy of a drug or treatment. Some methods further comprise performing a clinical trial in which a haplotype pattern that is associated with the differential relative allelic expression pattern is further analyzed to determine if the haplotype pattern is also associated with an adverse response to a drug or treatment. Some methods further comprise diagnosing a patient, wherein the presence or absence of a phenotypic trait is determined from presence or absence of a haplotype pattern that is associated with the differential relative allelic expression pattern. In some methods, the phenotypic trait is one or more of a disease state, susceptibility to a disease, resistance to a disease, or response to a drug.

In some methods, the differential relative allelic expression pattern is determined by hybridizing mRNA or cDNA to a probe array. In some methods, the differential relative allelic expression pattern is determined by performing a single base extension reaction using a primer having a 3′ end that hybridizes adjacent to a polymorphic site in the coding region of the gene. In some methods, the differential relative allelic expression pattern is determined by sequencing RNA transcripts or nucleic acids derived therefrom. In some methods, the differential relative allelic expression pattern is determined by allele-specific PCR amplification. In some methods, the differential relative allelic expression pattern is determined by analyzing amino acid differences in proteins expressed from different alleles of the same gene.

Some methods further comprise determining whether expressed genes are partially or completely within or proximate to the haplotype block that contains one or more haplotype patterns associated with the differential relative allelic expression pattern. In some methods, an expressed gene is located partially or completely within the haplotype block that contains one or more haplotype patterns associated with the differential relative allelic expression pattern and the method further comprises identifying an agent that alters the differential relative allelic expression pattern. In some methods, the agent alters the differential relative allelic expression pattern by interacting with the protein encoded by the expressed gene. In some methods, the agent alters the differential relative allelic expression pattern by interacting with the mRNA encoded by the expressed gene. In some methods, the agent alters the differential relative allelic expression pattern by binding to an entity that interacts with the protein encoded by the expressed gene. In some methods, the agent alters the differential relative allelic expression pattern by binding to an entity that interacts with the mRNA encoded by the expressed gene. In some methods, the agent alters the differential relative allelic expression pattern by inhibiting or stimulating, either directly or indirectly, the transcription of the expressed gene. In some methods, the agent alters the differential relative allelic expression pattern by inhibiting or stimulating, either directly or indirectly, the translation of the mRNA encoded by the expressed gene. In some methods, the agent alters the differential relative allelic expression pattern by disrupting the activity of the protein encoded by the expressed gene. In some methods, the agent alters the differential relative allelic expression pattern by disrupting the binding of the protein encoded by the expressed gene to DNA. In some methods, the cells are isolated from a tissue selected from the list comprising blood, liver, brain, skin, kidney, breast, prostate, colon, muscle, nerve, lung, heart, stomach, connective tissue, bone marrow, and tumor tissue.

In some methods, one or more haplotype patterns that are associated with the differential relative allelic expression patterns of the gene are identified, and the one or more haplotype patterns are also associated with the differential relative allelic expression pattern of at least one other gene. In some methods, a differential allelic expression pattern is determined for a plurality of genes, and step (b) is performed for each gene that exhibits a differential relative allelic expression pattern. In some methods, a plurality of haplotype patterns located in different haplotype blocks that are associated with the differential relative allelic expression pattern of the gene are identified. In some methods, a plurality of haplotype patterns, at least two of which are located in the same haplotype block, are identified and that are associated with the differential relative allelic expression pattern of the gene. In some methods, a plurality of haplotype patterns that cumulatively associate with the differential relative allelic expression pattern of the gene are identified. In some methods, a plurality of haplotype patterns located in different haplotype blocks that are associated with differential relative allelic expression patterns of a plurality of different genes including the gene are identified. In some methods, a plurality of haplotype patterns, at least two of which are located in the same haplotype block, and that are associated with differential relative allelic expression patterns of a plurality of different genes including the gene are identified. In some methods, a plurality of haplotype patterns that cumulatively associate with differential relative allelic expression patterns of a plurality of different genes including the gene are identified.

In some methods, no single polymorphic form in the haplotype block is solely responsible for causing the differential relative allelic expression patterns of the gene. In some methods, the haplotype pattern is associated with differential gene expression and one of the polymorphic forms of the haplotype pattern is not directly involved in differential expresssion and the method further comprises using the polymorphic form as a marker to detect a second polymorphic form that is directly involved in the differential relative allelic expression pattern. In some methods, a second gene is identified that overlaps at least in part with the haplotype block, wherein alteration of the expression level of the second gene or the function of its gene product alters the differential relative allelic expression pattern.

In some methods, one or more haplotype patterns associated with the differential relative allelic expression pattern of the gene are identified, and the method further comprises scanning one or more haplotype blocks containing the one or more haplotype patterns associated with the differential relative allelic expression pattern for the presence of expressed genes.

In some methods, an associated haplotype pattern that is associated with the differential relative allelic expression pattern of the gene is identified, and the method further comprises the step of performing an association analysis, wherein the test group is a subset of samples that exhibit the differential relative allelic expression pattern of the gene and have the associated haplotype pattern and the control group is a subset of samples that do not exhibit the differential relative allelic expression pattern of the gene and have the associated haplotype pattern, wherein a second associated haplotype pattern that is associated with the differential relative allelic expression pattern of the gene is identified.

In some methods, an associated haplotype pattern that is associated with the differential relative allelic expression pattern of the gene is identified, and the method further comprises the step of performing an association analysis, wherein a first group is a subset of samples that exhibits a first ratio of reference:alternate expression levels and has the associated haplotype pattern and a second group is a subset of samples that exhibits a second distinct ratio of reference:alternate expression levels and has the associated haplotype pattern, and further wherein a second associated haplotype pattern that is associated with the difference in magnitude of the first and second ratios is identified.

The invention further provides methods of characterizing a gene. These methods involve determining a differential relative allelic expression pattern of at least two alleles of the gene from samples containing diploid cells from a plurality of individuals of the same species, where the cells are heterozygous for said gene. One then determines whether the differential relative allelic expression pattern of the gene is associated with a polymorphic form at a polymorphic site outside the gene and regulatory regions that control the transcription thereof.

### BRIEF DESCRIPTION OF THE DRAWINGS

FIG. 1 is an illustrative example of SNPs that are inherited as units within haplotype blocks.

FIG. 2 illustrates the process of choosing PCR primer pairs to amplify transcribed SNPs.

FIG. 3 illustrates RNA and DNA isolation from tissue samples from 12 individuals. Sequences encoding transcribed SNPs were amplified from the RNA and DNA samples from each individual and were hybridized to high density oligonucleotide arrays.

FIGS. 4A-D illustrate experimental results from samples taken from Individuals One and Four, with each point representing a single transcribed SNP. FIG. 4A illustrates plotting DNA versus DNA duplicate p-hat values from a single individual (Individual One), and RNA versus RNA duplicate p-hat values from the same individual. FIG. 4B illustrates the average of the duplicate RNA p-hat values plotted against the average of the duplicate DNA p-hat values in the sample from Individual One. FIG. 4C illustrates the average of the duplicate RNA p-hat values plotted against average of the duplicate DNA p-hat values in the sample from Individual Four for the same set of SNPs as shown for Individual One in FIG. 4B.

FIGS. 5A-D illustrate the verification of data from array hybridization by real-time PCR. FIG. 5A illustrates that allele frequency can be calculated by real-time PCR. FIG. 5B illustrates allele frequencies from RNA samples from a KCNJ6 gene heterozygote measured by real-time PCR (asterisks) plotted against a standard curve generated by the data in FIG. 5A (diamonds). FIG. 5C illustrates that genes that do not display differential expression patterns between two alleles, such as the ADARB 1 gene, can also be detected by real-time PCR. FIG. 5D illustrates that a gene, HS3ST1, that demonstrates a differential relative allelic expression pattern based on an array data analysis also demonstrates a differential relative allelic expression pattern when analyzed with real-time PCR analysis.

FIG. 6 illustrates that for Individual One, 783 SNPs are heterozygous and expressed.

FIG. 7 illustrates two examples of haplotype defining SNPs in which 5 or more heterozygotes demonstrate similar differential relative allelic expression patterns such that the same allele is consistently expressed at a higher level.

### DETAILED DESCRIPTION OF THE INVENTION

Definitions

The term “SNP” or “single nucleotide polymorphism” refers to a genetic variation between individual DNA strands at a single nitrogenous base position in the DNA.

Reference to DNA includes derivatives of DNA including but not limited to amplicons, RNA transcripts, and cDNA, unless otherwise apparent from the context. The term “polymorphic form” refers to the identity of a nucleotide or the sequence of a plurality of nucleotides that occur at a position that is variable in a genome. When used in reference to a SNP, “polymorphic form” refers to the nucleotide identity of the nitrogenous base that occupies the SNP location.

The term “SNP location” refers to the position in a genome at which a SNP occurs.

The term “biallelic SNP” refers to a SNP that occurs in two polymorphic forms.

The term “triallelic SNP” refers to a SNP that occurs in three polymorphic forms.

The term “common polymorphic forms” refers to sequence variants, including SNPs, insertions, deletions, and other sequence variations that occur at a frequency of more than 0.05 in genomes of the same species. The term “common polymorphic site” refers to a site in a genome that may contain two or more common polymorphic forms. The term “common SNP” refers to a SNP that has at least two polymorphic forms, each of which occurs at a frequency of more than 0.05 in genomes of the same species. The term “rare SNP” refers to a SNP having only one polymorphic form occurring at a frequency of more than 0.05 in genomes of the same species.

The term “haplotype block” refers to a region of a chromosome that contains one or more polymorphic sites (e.g., 1-10) that tend to be inherited together. In other words, combinations of polymorphic forms at the polymorphic sites within a block cosegregate in a population more frequently than combinations of polymorphic sites that occur in different haplotype blocks. Polymorphic sites within a haplotype block tend to be in linkage disequilibrium with each other. Often, the polymorphic sites that define a haplotype block are common polymorphic sites. Some haplotype blocks contain a polymorphic site that does not cosegregate with adjacent polymorphic sites in a population of individuals.

The term “haplotype defining polymorphic site” refers to a polymorphic site whose variant form allows one to predict the identity of other variant forms occupying other polymorphic sites in the same haplotype block. Often, a haplotype defining polymorphic site is also a common polymorphic site.

The term “haplotype pattern” refers to a combination of polymorphic forms that occupy polymorphic sites, usually SNPs, in a haplotype block on a single DNA strand. For example, the combination of variant forms that occupy all the polymorphisms within a particular haplotype block on a single strand of nucleic acid is collectively referred to as a haplotype pattern of that particular haplotype block. Often, the polymorphic sites that define a haplotype pattern are common polymorphic sites. In certain embodiments, 80% of the haplotype patterns found in a given haplotype block in a sample of 20 or more genomes are one of only four or fewer distinct haplotype patterns.

A “transcribed polymorphism” occurs within a transcribed region of a gene.

A “differential relative allelic expression pattern” refers to the relative expression levels of one allele of a gene (arbitrarily labeled as the “reference allele”) as compared to a different allele of the same gene (arbitrarily labeled as the “alternate allele”) when both alleles are present in the same diploid cell. For a biallelic gene three allelic expression patterns may occur. In the first, the reference allele is expressed at a higher level than the alternate allele (the “reference>alternate pattern”). In the second, the alternate allele is expressed at a higher level than the reference allele (the “reference<alternate pattern”). In the third both alleles are expressed at the same level.

The term “differentially expressed gene” refers to a gene that has multiple alleles, at least one of which differs in expression level compared to at least one other allele when both alleles are present in the same diploid cell.

The term “obtained directly from an organism” means not cultured.

The term “individual” refers to a specific single organism, such as a single animal, human, insect, bacterium, or other life form.

The term “linkage disequilibrium” refers to the preferential segregation of a particular polymorphic form with another polymorphic form at a different chromosomal location more frequently than expected by chance. Linkage disequilibrium can also refer to a situation in which a phenotypic trait displays preferential segregation with a particular polymorphic form or another phenotypic trait more frequently than expected by chance.

The term “linkage equilibrium” refers to a random pattern of segregation of a particular polymorphic form with another polymorphic form at a different chromosomal location. Linkage equilibrium can also refer to a situation in which a phenotypic trait displays a random pattern of segregation with a particular polymorphic form or another phenotypic trait.

A polymorphic site is proximal to a gene if it occurs within the intergenic region between the transcribed region of the gene and an adjacent gene. Usually, proximal implies that the polymorphic site occurs closer to the transcribed region of the particular gene that that of an adjacent gene. Typically, proximal implies that a polymorphic site is within 50 kb, and preferably within 10 kb of the transcribed region. Polymorphic sites not occurring in proximal regions as defined above are said to occur in regions that are distal to the gene.

The term “comprising” indicates that other elements can be present besides those explicitly stated.

The term “agent” describes any molecule such as a protein or small molecule that has the capability of altering, mimicking or masking, either directly or indirectly, the physiological function of amidentified gene or gene product.

Specific binding between two entities means a mutual affinity of at least 10⁶ M⁻¹, and usually at least 10⁷ or 10⁸ M-1. The two entities also usually have at least 10-fold greater affinity for each other than the affinity of either entity for an irrelevant control.

“Statistically significant” means significant at a p value<0.05.

“Substantially all regions of the genome” means at least 95% of unique sequences in the genome.

I. General

The invention provides methods of identifying the genetic basis of differential relative allelic expression patterns. The present invention provides the insight that the genetic basis largely resides not in isolated polymorphisms occurring within regions such as promoters and enhancers controlling expression of a gene, but rather in haplotype blocks and patterns that contain at least one polymorphic site and usually multiple polymorphic sites. The invention provides the further insight that haplotype patterns associated with differential relative allelic expression patterns can occur not simply proximal to the gene whose alleles are differentially expressed, but at widely dispersed distal locations throughout the genome as well. In addition, the invention provides the further insight that polymorphisms in haplotype patterns that are associated with differential relative allelic expression patterns may be directly involved in the differential relative allelic expression patterns (a “functional polymorphism”), or may be in linkage disequilibrium with one or more functional polymorphisms. Although a functional polymorphism may be detected directly, in some embodiments, such a polymorphism is detected indirectly by assaying for another polymorphism or a haplotype pattern with which the functional polymorphism is in linkage disequilibrium.

Although an understanding of mechanism is not essential for practice of the invention, it is believed that multiple polymorphic sites in proximity to an allele can affect expression of an allele by influencing chromatin formation and accessibility of the allele to transcription factors through the alteration of the aggregate scaffolding of proteins that are bound to each respective allele. Other polymorphic sites that are proximal to a gene and are associated with differential relative allelic expression patterns are not causatively associated with the patterns but are in linkage disequilibrium with polymorphic sites that are causatively associated with the patterns (i.e. functional polymorphisms). Haplotype patterns at distant chromosomal locations can influence differential expression of alleles in combination with haplotype patterns proximate to the alleles. For example, different variants of transcription factors can interact differently with variant alleles of other genes to cause differential expression of the alleles. Other pathways that may also be involved in differential relative allelic expression patterns include, but are not limited to, transcriptional regulation pathways (e.g. involving enhancer or other regulatory sequences), post-transcriptional modification pathways (e.g. splicing), mRNA degradation pathways, translational regulation pathways, post-translational modification pathways (e.g. phosphorylation, methylation and glycosylation), and protein degradation pathways.

The methods of the invention work by determining the relative expression levels of alleles of the same gene in different individuals. When different alleles of the same gene are expressed at different levels in an individual, this is known as a differential relative allelic expression pattern. These same individuals are genotyped to determine haplotype patterns at one or more haplotype blocks throughout the genome. Preferably, haplotype patterns at all or substantially all haplotype blocks in the genome are genotyped for each individual. Analyzing haplotype patterns at all haplotype blocks in a genome results in analyzing the entire genome of the individual for associated haplotype patterns. Differential relative allelic expression patterns are then analyzed for association with haplotype patterns for the population of individuals.

Haplotype patterns associated with differential relative allelic expression patterns are useful for a variety of purposes. These haplotype patterns may be used in further analysis to associate the haplotype patterns with phenotypic traits including, but not limited to, resistance or susceptibility to a disease, or response to a drug or other medical treatment. This type of analysis is particularly useful for multi-locus associations between differential relative allelic expression patterns of a gene and various haplotype patterns. Haplotype patterns associated with differential relative allelic expression patterns can be used to diagnose diseases or other phenotypes associated with the patterns. The haplotype patterns may also be used to perform clinical trials on a pharmaceutical composition on populations of patients. The haplotype patterns may also be used to identify drug targets for treatment of diseases associated with differential relative allelic expression patterns.

II. Sample Preparation

Cells are isolated from individuals, such as humans. The cells can be from any tissue in the organism. For instance, blood is drawn from humans and lymphocytes are separated from plasma using standard procedures. Alternatively, cells are removed from other tissue or organ types such as liver, brain, skin, kidney, breast, prostate, colon, muscle, nerve, lung, heart, the gastrointestinal tract, connective tissue, bone marrow, benign or cancerous tumor, and others using standard techniques. Cells can be used directly from an individual or can be cultured. Total RNA or messenger RNA (mRNA) is purified from the cells, in some methods without the cells being cultured or propagated in vitro, using standard techniques provided in sources such as Sambrook, et al., Molecular Cloning: A Laboratory Manual (Cold Spring Harbor Laboratory, New York) (1989). In some instances, cells (e.g. lymphoblasts) or tissues (e.g. liver, brain, skin, kidney, breast, prostate, colon, muscle, nerve, lung, heart, the gastrointestinal tract, connective tissue, bone marrow, benign or cancerous tumor) may be cultured prior to use by methods well known in the art.

In some instances, individuals who are either healthy or alternatively are experiencing the same disease state are selected. For example, blood is drawn from a plurality of healthy human subjects. mRNA is then purified from the cells and analyzed for the presence of mRNA transcripts from different alleles of the same gene that are present in different amounts in each individual. Alternatively, protein can be isolated from the cells or tissue for detection of differential expression at the protein level. Genomic DNA can be isolated from the same cells for analysis of polymorphic sites.

RNA, DNA, and proteins are isolated according to conventional procedures, such as those described in Sambrook, et al., Molecular Cloning: A Laboratory Manual (Cold Spring Harbor Laboratory, New York) (1989), and Ausubel, et al, Current Protocols in Molecular Biology (John Wiley and Sons, New York) (1997), each of which is incorporated by reference.

The nucleic acids used for genotyping polymorphisms can be amplified. Detailed protocols for PCR are provided in PCR Protocols, A Guide to Methods and Applications, Innis et al., Academic Press, Inc. N.Y., (1990). Other suitable amplification methods include the ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)). Techniques to optimize the amplification of long sequences can be used. Such techniques work well on genomic sequences. The methods disclosed in pending US patent applications U.S. Ser. No. 10/042,406, filed Jan. 9, 2002 entitled “Algorithms for Selection of Primer Pairs”; and U.S. Ser. No. 10/042,492, filed Jan. 9, 2002, entitled “Methods for Amplification of Nucleic Acids”, both assigned to the assignee of the present invention, are particularly suitable for amplifying genomic DNA for use in the methods of the present invention.

The nucleic acids can be labeled to facilitate detection in subsequent steps. Labeling can be carried out during an amplification reaction by incorporating one or more labeled nucleotide triphosphates and/or one or more labeled primers into the amplified sequence. The nucleic acids can be labeled following amplification, for example, by covalent attachment of one or more detectable groups. Any detectable group known can be used, for example, fluorescent groups, ligands and/or radioactive groups.

Amplified sequences can be subjected to other post-amplification treatments either before or after labeling. For example, in some instances the DNA is fragmented prior to hybridization with an oligonucleotide array. Fragmentation of the nucleic acids generally can be carried out, for example, by subjecting the amplified nucleic acids to shear forces by forcing the nucleic acid containing fluid sample through a narrow aperture or digesting the PCR product with a nuclease enzyme. One example of a suitable nuclease enzyme is DNase I.

RNA (e.g., mRNA) is purified from cells from the same individual from which DNA is obtained in the methods of the preceding paragraphs. A section of the RNA from each gene that contains the transcribed polymorphism is amplified with a primer pair by RT-PCR such that the RT-PCR product contains the known polymorphism. For genes that are heterozygous for a transcribed polymorphism, the same primer set generates RT-PCR products that differ in sequence by at least the two polymorphic forms of the transcribed polymorphism. Optionally, the same primer pairs are used to amplify transcribed polymorphism sequences from genomic DNA and RNA samples.

### III. Differential Relative Allelic Expression Patterns

### A. General

In a diploid cell there are generally two copies of each gene in the genome contained in the cell. In many instances distinct alleles of a gene are expressed at the same level in a cell; in other instances two or more alleles are expressed at different levels in a cell. Such differential relative allelic expression patterns of a gene can be measured if any sequence differences between the two alleles such as polymorphisms (e.g., SNPs) fall within the transcribed region of the gene. For biallelic polymorphisms, for example, one polymorphic form of the transcribed polymorphism is referred to as the “reference allele”, and the other polymorphic form of the transcribed polymorphism is referred to as the “alternate allele”. mRNA transcribed from each allele is identified in a sequence-specific fashion so that the amount of mRNA transcribed from one allele may be compared to the amount of mRNA transcribed from the other allele when both alleles are present in the same diploid cell.

### B. Probe Array Methods of Measuring Differential Relative Allelic Expression Patterns

In some methods, presence of allelic variation at the DNA level and differential expression of alleles at the mRNA level are both determined by hybridization to an array, optionally, simultaneously. See Chee, U.S. Pat. No. 6,368,799. Genomic DNA or PCR products generated therefrom are hybridized to an array to determine the presence of heterozygous polymorphic forms of a gene. RNA, RT-PCR products generated therefrom, or cDNA generated therefrom are also hybridized to an array to determine if different alleles of a gene are expressed at different levels. The two hybridizations can be performed simultaneously on the same array if genomic DNA and mRNA are differentially labeled. The genomic analysis identifies one or more genes that are heterozygous for a polymorphism occurring within a transcribed region of a gene. The RNA analysis determines the relative amount of different polymorphic forms of the transcripts of genes that are identified as heterozygous by the genomic analysis.

Genotyping by probe array methods is usually performed after the location and nature of polymorphic forms present at a site have already been determined. The availability of this information allows sets of probes to be designed for specific identification of the known polymorphic forms. In the simplest form of analysis, a biallelic SNP or other biallelic polymorphic form is characterized using a pair of allele-specific probes respectively hybridizing to the two polymorphic forms. However, the analysis is more accurate using specialized arrays of probes based on the respective polymorphic forms. Often the probes on an array are tiled, which refers to the use of groups of related immobilized probes, some of which show perfect complementarity to a reference sequence and others of which show mismatches from the reference sequence (for example, see WO95/11985). A typical array for analyzing a known biallelic SNP contains two groups of probes based on two sequences constituting the respective reference and alternate polymorphic forms.

The first group of probes includes at least a first set of one or more probes which span the polymorphic site and are exactly complementary to one of the polymorphic forms (e.g., “reference” polymorphic form). The group of probes can also contain second, third and fourth additional sets of probes which contain probes identical to probes in the first probe set except at one position referred to as an interrogation position. When such a probe group is hybridized with the polymorphic form constituting the reference sequence, all probes in the first probe set exhibit perfect hybridization and all of the probes in the other probe sets exhibit background hybridization patterns due to mismatches.

When such a probe group is hybridized with the other polymorphic form, a different pattern is obtained. That is, all but one probe in the array show a mismatch to the target and produce only background hybridization. The one probe that exhibits perfect hybridization is a probe from the second, third or fourth probe sets whose interrogation position aligns with the polymorphic site and is occupied by a base complementary to the other polymorphic form.

When the probe group is hybridized with a heterozygous sample in which both polymorphic forms are present, the patterns for the homozygous polymorphic forms are superimposed. Thus, the probe group exhibits distinct and characteristic hybridization patterns depending on which polymorphic forms are present and whether an individual is homozygous or heterozygous for the biallelic polymorphic form.

Typically, an array also contains a second group of probes tiled using the same principles as the first group but with the second probe set spanning the polymorphic site and showing perfect complementary to the other polymorphic form (e.g., “alternate” polymorphic form”). Hybridization of the second probe group to homozygous or heterozygous target sequences yields a hybridization pattern that is complementary to that of the first group. By analyzing the hybridization patterns from both probe groups, one can determine with high accuracy which polymorphic form(s) are present in an individual.

The same probe arrays that are used for analyzing polymorphic forms in genomic DNA can be used for analyzing polymorphic forms of transcripts. The hybridization patterns of the probe arrays are analyzed in the same manner for genomic DNA targets, genomic DNA-derived targets such as PCR products, RNA targets, and RNA-derived targets such as RT-PCR products or cDNA. For example, DNA copies of transcripts may be generated by RT-PCR and then hybridized to the array. Comparison of the hybridization intensities of the first probe group that are perfectly matched with one polymorphic form to the hybridization intensities of the second probe group that are perfectly matched with the second polymorphic form indicates the relative proportions of the polymorphic forms of the transcript.

Relative allele concentration is the ratio of the abundance of a particular transcribed polymorphic form to the abundance of all transcribed forms of the polymorphism (e.g., SNP), and may be expressed by the equation: (cR/CR+cA), where CR is the concentration of the reference allele and CA is the concentration of the alternate allele. The sum of the relative allele concentrations for all of the polymorphic forms of a given polymorphism is one. For example, when genomic DNA is heterozygous at a SNP location, the ratio of DNA fragments containing one polymorphic form of the SNP to fragments containing the other polymorphic form of the SNP is 1:1, and the relative allele concentration of each polymorphic form of the SNP is 0.5 (0.5+0.5=1). In a genomic DNA sample that is homozygous for either polymorphic form of a SNP, the relative allele concentrations for the reference and alternate alleles should be 0 and 1.0 or 1.0 and 0, depending on which polymorphic form is present in both copies of the gene.

Like relative allele frequencies for DNA samples, the sum of the relative allele frequencies for each polymorphic form of the transcribed SNP (i.e., expressed as mRNA) encoded by the DNA also add together to equal 1.0. For example, when the two alleles of the gene are expressed at approximately equal levels, then each polymorphic form of RNA encoding the transcribed SNP has a relative allele frequency of approximately 0.5. If the two alleles of the gene are expressed at different levels then there are unequal concentrations of each mRNA transcript, and thus alleles containing different polymorphic forms of the transcribed SNP have different relative allele frequencies.

To determine whether variant forms of a transcribed polymorphism display differential relative allelic expression levels, the relative allele frequencies of the polymorphic forms in the DNA encoding the transcribed polymorphism may be compared to the relative allele frequencies of the transcribed polymorphic forms themselves. If the relative allele frequencies of the transcribed polymorphisms in the DNA sample are substantially similar to the relative allele frequencies for the transcribed polymorphisms in the RNA sample, then it is unlikely that the transcribed polymorphisms are differentially expressed. Alternatively, if the relative allele frequencies of the transcribed polymorphisms in the DNA sample are substantially different from the relative allele frequencies for the transcribed polymorphisms in the RNA sample, then it is likely that the transcribed polymorphisms are differentially expressed.

In certain embodiments, the relative allele frequency may be estimated using a measure known as “p-hat”, which is derived from experiments that indirectly measure the frequencies of each allele. In certain embodiments, p-hat is the relative concentration of the reference allele over the total, but may also be calculated as the relative concentration of the alternate allele over the total. For estimated relative allele concentrations in a DNA sample, the value is referred to as “DNA p-hat”, and in an RNA sample (or a cDNA sample derived from RNA) it is referred to as “RNA p-hat”. Theoretically, the DNA p-hat value for each polymorphic form in a heterozygote should be 0.5, but since the p-hat value is a value based on experimental measurements it may vary somewhat due to various criteria related to experimental design. In one embodiment, when the DNA p-hat value of a polymorphic form of a transcribed SNP is between approximately 0.4 and 0.7 as determined from analysis of genomic DNA, the genomic DNA is considered to be heterozygous for the two forms of the transcribed SNP.

DNA and RNA p-hat values for a first polymorphic form can be compared to DNA and RNA p-hat values for a second polymorphic form at the same polymorphic site to determine whether or not the first and second polymorphic forms are differentially expressed. For example, if a polymorphic form of a transcribed SNP in a gene has a DNA p-hat value of approximately 0.4-0.7 and the RNA p-hat value of transcript containing the same polymorphic form of the transcribed SNP is within approximately 0.1 of the value of the DNA p-hat, this result indicates that the different alleles of the gene are transcribed in the same cell in approximately equal amounts. Alternatively, if a polymorphic form of a transcribed SNP in a gene has a DNA p-hat value of approximately 0.4-0.7 and the RNA p-hat value of transcript containing the same polymorphic form of the transcribed SNP differs from its DNA p-hat by 0.1 or more, this result indicates that the different alleles of the gene are transcribed in the same cell at different levels. This second result is indicative of a differential relative allelic expression pattern.

Cell samples are obtained from a plurality of individuals and are analyzed at one or more transcribed SNPs. Preferably at least 100, 1,000, 10,000, 100,000, or 1,000,000 transcribed SNPs are analyzed. In certain embodiments, each transcribed SNP analyzed is located in a different gene; in other embodiments more than one transcribed SNP may be analyzed in a single gene. In certain embodiments, only common SNPs are assayed; in other embodiments, both common and rare SNPs are assayed. Some genes display differential relative allelic expression patterns in all individuals. Some genes display differential relative allelic expression patterns in some individuals but not others. Some genes display differential relative allelic expression patterns in which the reference allele is transcribed at a higher level than the alternate allele in all or a subset of individuals, or alternatively the reference allele is transcribed at a lower level than the alternate allele in all or a subset of individuals. Some genes do not display differential relative allelic expression patterns in any observed individuals. Some genes display differential relative allelic expression patterns only in certain tissue types or stages of development.

Similar differential relative allelic expression patterns occur when one of the alleles is expressed at a higher level than the other allele in two or more individuals that are heterozygous for the same alleles, but the ratio of the expression patterns of the two alleles is variable (that is, how much higher the expression of one is over the other is variable). Identical differential relative allelic expression patterns occur when one allele is expressed at a higher level than a second allele in two or more samples and the ratio of the expression patterns of the two alleles in those samples is identical within a defined limit, such as 1.7+0.1:1.

### C. Single Base Primer Extension Methods of Measuring Differential Relative Allelic Expression Patterns

Another method of analyzing differential relative allelic expression patterns relies on single base extension of a primer that is designed to anneal immediately adjacent to the position of a known polymorphic site in a target nucleic acid. This method is generally used only when the position of a polymorphic site is known because the primer must anneal to a complementary sequence immediately adjacent to the polymorphic site. The primer anneals adjacent to the polymorphic site in either target DNA or RNA molecules. Target nucleic acids are purified from cells or tissue or alternatively nucleic acids are amplified by PCR in which the template comprises nucleic acids purified from cells or tissue. Alternatively the target nucleic acid may be a clone of a gene propagated in a host or a transcript of the clone. In addition to primer and target nucleic acid, DNA polymerase and a labeled nucleotide or a plurality of differentially labeled nucleotides of different types are added to the reaction. The polymerase adds to the primer only a labeled nucleotide that is complementary to the position in the target nucleic acid immediately adjacent to the nucleotide at the 3′ end of the annealed primer. This position is the polymorphic site. The reaction is then analyzed to determine if a labeled nucleotide has been added to the primer.

If, for example, a biallelic polymorphic site contains either an Adenine or Cytosine, differentially fluorescently labeled Guanine and Thymine nucleotides are added to the reaction. The primer anneals to the target nucleic acid immediately adjacent to the polymorphic site. If the target nucleic acid is a genomic DNA sample from a diploid cell, it may be homozygous for Adenine, homozygous for Cytosine, or heterozygous; the resulting primers after extension by DNA polymerase therefore contain only labeled Thymine, only labeled Guanine, or labeled Thymine and labeled Guanine in approximately equal amounts, respectively. For examples, see Soderlund et al., U.S. Pat. No. 6,013,431 and Yan et al., Science 2002 Aug. 16;297(5584):1143. If the target nucleic acid is an mRNA transcript or RT-PCR product derived therefrom from a diploid cell that is heterozygous for a given polymorphic site, the respective amounts of primer containing labeled Guanine and labeled Thymine depend on the relative expression levels of the two alleles of the gene that contain the different SNPs. If the expression level is approximately the same for both alleles then the ratio of Guanine-labeled primer to Thymine-labeled primer is approximately 1:1. If the expression level of each allele is different between the two alleles then the ratio is not 1:1 and this result is indicative of a differential relative allelic expression pattern.

### D. Allele-Specific PCR Amplification Methods of Measuring Differential Relative Allelic Expression Patterns

Another method of determining differential relative allelic expression patterns is the selective PCR amplification of different alleles of a gene. In this method PCR primers are designed to anneal or to not anneal to a template at a given temperature depending on the sequence of the template. For example, PCR primers to detect a biallelic polymorphism are designed so that a first primer anneals to the sense strand of the template in a non-polymorphic region of the gene and a second primer is designed to anneal to the antisense strand of the gene at the polymorphic site. The second primer is designed such that at a given hybridization temperature it only anneals if the first of the two polymorphic forms is present in the template strand. A PCR reaction is performed in which the nucleic acid sequence between the two binding sites will only be amplified if the first of the two polymorphic forms is present in the template strand. In a separate PCR reaction the same template is included along with the same first primer, however a third primer is included in the reaction rather than the second primer. The third primer is designed such that at a given hybridization temperature it only anneals if the second of the two polymorphic forms is present in the template strand, thereby facilitating PCR amplification of only nucleic acids containing the second of the two polymorphic forms.

When the template nucleic acid is a genomic DNA sample from a diploid cell, it may be homozygous for the first polymorphic form, homozygous for the second polymorphic form, or heterozygous. When the template is homozygous for the first polymorphic form a PCR product is generated only in the reaction containing the first and second primers but not the reaction containing the first and third primers. When the template is homozygous for the second polymorphic form a PCR product is generated only in the reaction containing the first and third primers but not the reaction containing the first and second primers. When the template is heterozygous, PCR products are generated in both reactions. For example, see Faas et al., Blood 1995 Feb. 1;85(3):829-32. 100781 When the template is mRNA isolated from heterozygous cells and RT-PCR is performed, or if the template is the DNA product of such an RT-PCR reaction, the relative amounts of the two PCR products depends on the relative transcription levels of the two alleles if the polymorphic forms of each allele occur at a transcribed SNP position. When the expression level is approximately the same for both alleles then the ratio of PCR products is approximately 1:1. If the expression level of each allele is different between the two alleles then the ratio of PCR products is not approximately 1:1 and this result is indicative of a differential relative allelic expression pattern.

### E. Protein Analysis Methods of Measuring Differential Relative Allelic Expression Patterns

Differential relative allelic expression patterns can also be determined from different amounts of protein variants encoded by separate alleles of a gene, if the different alleles code for proteins with a different amino acid sequence. For example, protein is isolated from cells or tissue and subjected to immunoblotting by monoclonal antibodies that differentially recognize polymorphic forms of proteins that possess amino acid substitutions encoded by different alleles of the gene. For example, see Cohen et al., J Clin Endocrinol Metab 1996 October;81(10):3505-12. Polymorphic forms of proteins can also be detected using mass spectrometry or protein truncation assays. For examples see Klose et al., Nat Genet 2002 April;30(4):385-93 and Kinzler et al., U.S. Pat. No. 5,709,998.

When the expression levels of two different alleles of a gene that encodes a particular protein in a heterozygous diploid cell are approximately the same, then the ratio of the two forms of the protein in a sample is usually approximately 1:1. When the expression levels are different between the two alleles then the ratio of the two forms of the protein in a sample is usually not approximately 1:1; this result is indicative of a differential relative allelic expression pattern.

Whereas differential relative allelic expression patterns of mRNAs give mRNA p-hat values, those of proteins give protein p-hat values. Other methods of determining differential relative allelic expression patterns may also be performed. The invention is not limited to those methods of determining differential relative allelic expression patterns listed above.

### IV. Methods of Genotyping SNPs

The following methods can be used at two stages in the procedure. First, the methods can be used to identify heterozygous polymorphisms occurring within transcribed regions to be used in determining allelic expression levels. As indicated above, such is preferably performed in combination with determining allelic expression levels but can also be performed separately. Second, the methods are used to determine polymorphic forms occupying polymorphic sites throughout the genome for use in correlating haplotype patterns with differential expression.

Polymorphisms can be genotyped by direct sequencing of DNA. The DNA may be amplified prior to direct sequencing. Hybridization techniques can also be employed to identify haplotype patterns or haplotype-defining SNPs. For example, in certain embodiments of the present invention, high density oligonucleotide arrays may be utilized for the detection of SNPs, such as those commercially available from Affymetrix, Inc. (Santa Clara, Calif.).

Invader™ technology available from Third Wave Technologies, Inc., Madison, Wis. can be used to analyze polymorphisms without amplification (see Hessner, et al., Clinical Chemistry 46(8):1051-56 (2000) and Hall, et al., PNAS 97(15):8272-77 (2000)). Two short DNA probes hybridize to a target nucleic acid to form a structure recognized by a nuclease enzyme. For SNP analysis, two separate reactions are run, one for each SNP variant. If one of the probes is complementary to the sequence, the nuclease cleaves it to release a short DNA fragment termed a “flap”. The flap binds to a fluorescently-labeled probe and forms another structure recognized by a nuclease enzyme. When the enzyme cleaves the labeled probe, the probe emits a detectable fluorescence signal thereby indicating which SNP variant is present.

Rolling circle amplification utilizes an oligonucleotide complementary to a circular DNA template to produce an amplified signal (see, for example, Lizardi, et al., Nature Genetics 19(3):225-32 (1998); and Zhong, et al., PNAS 98(7):3940-45 (2001)). Extension of the oligonucleotide results in the production of multiple copies of the circular template in a long concatamer. Typically, detectable labels are incorporated into the extended oligonucleotide during the extension reaction. The extension reaction can be allowed to proceed until a detectable amount of extension product is synthesized.

Another technique suitable for the analysis of polymorphisms is the Taqman™ assay (see, e.g., Arnold, et al., BioTechniques 25(1):98-106 (1998); and Becker, et al., Hum. Gene Ther. 10:2559-66 (1999)). A target DNA containing a SNP is amplified in the presence of a probe molecule that hybridizes to the SNP site. The probe molecule contains both a fluorescent reporter-labeled nucleotide at the 5′ end and a quencher-labeled nucleotide at the 3′ end. The probe sequence is selected so that the nucleotide in the probe that aligns with the SNP site in the target DNA is as near as possible to the center of the probe to maximize the difference in melting temperature between the correct match probe and the mismatch probe. As the PCR reaction is conducted, the correct match probe hybridizes to the SNP site in the target DNA and is digested by the Taq polymerase used in the PCR assay. This digestion results in physically separating the fluorescently labeled nucleotide from the quencher with a concomitant increase in fluorescence. The mismatch probe does not remain hybridized during the elongation portion of the PCR reaction and is therefore not digested and the fluorescently labeled nucleotide remains quenched.

Polymorphisms can also be analyzed by denaturing HPLC using a polystyrene-divinylbenzene reverse phase column and an ion-pairing mobile phase. A DNA segment containing a SNP is PCR amplified. After amplification, the PCR product is denatured by heating and mixed with a second denatured PCR product with a known nucleotide at the SNP position. The PCR products are annealed and are analyzed by HPLC at elevated temperature. The temperature is chosen to denature duplex molecules that are mismatched at the SNP location but not to denature those that are perfect matches. Under these conditions, heteroduplex molecules typically elute before homoduplex molecules. For example, see Kota, et al., Genome 44(4):523-28 (2001).

Polymorphisms can also be analyzed using solid phase amplification and microsequencing of the amplification product. Beads to which primers have been covalently attached are used to carry out amplification reactions. The primers are designed to include a recognition site for a Type II restriction enzyme. After amplification, which results in a PCR product attached to the bead, the product is digested with the restriction enzyme. Cleavage of the product with the restriction enzyme results in the production of a single stranded portion including the SNP site and a 3′-OH that can be extended to fill in the single stranded portion. Inclusion of ddNTPs in an extension reaction allows direct sequencing of the product. For example, see Shapero, et al., Genome Research 11(11): 1926-34 (2001).

### V. Association of Differential Relative Allelic Expression Patterns with Halotype Patterns

### A. General

The presence of differentially expressed heterozygous genes is first determined for one or more genes in a sample of cells obtained from one or more individuals using methods described in the preceding sections. The individuals are also genotyped at a collection of polymorphisms, preferably from throughout their genomes. The polymorphic forms present at the polymorphic sites are grouped into haplotype blocks and patterns, either prior or subsequent to the genotyping. The size of haplotype blocks associated with differential allelic expression depends on the method used to define the haplotype structure of a nucleic acid (e.g. a genome or portion thereof), and so may range from less than 5 kb to longer than 100 kb in length. Further, haplotype blocks and their constituent patterns may be defined such that all common SNPs are correlated with one another, or such a strict correlation may not be required. The polymorphic forms either individually or as haplotype patterns are then analyzed for an association with the differential relative allelic expression patterns for a particular gene that is differentially expressed. This process is repeated for each gene that exhibits a differential relative allelic expression pattern.

### B. Haplotype Pattern Determination for Samples

The determination of haplotype blocks in the human or other genome and characterization of which polymorphisms within them are haplotype-defining need be performed only once. There are many different ways to define haplotype blocks, and one preferred method is described in Patil, et al., “Blocks of Limited Haplotype Diversity Revealed by High-Resolution Scanning of Human Chromosome 21”, Science, 294:1719-1723 (2001). Once haplotype blocks for a DNA sequence (e.g. a portion or substantially all of a genome) have been defined, the haplotype patterns present in the haplotype blocks may be identified by 1) determining which polymorphic forms are present in each haplotype block on a single DNA strand, or 2) determining which polymorphic forms occupy the haplotype-defining polymorphisms in an individual. Both can be determined by the conventional genotyping procedures described previously.

In general, SNPs have been found to occur throughout the human genome approximately every 600 base pairs (Kruglyak and Nickerson, Nature Genet. 27:235 (2001), although most SNPs are rare SNPs. In general, the polymorphic form of a rare SNP is not predictive of the polymorphic form of other common SNPs located in the same haplotype block. By contrast, the polymorphic form of a common SNP is typically predictive of the polymorphic form of other common SNPs located in the same haplotype block. This is the case for all haplotype blocks that comprise more than one common SNP. For example, if a haplotype block contains more than one common SNP, the identity of one common SNP in the haplotype block may be predictive of the identity of another common SNP in the same haplotype block.

If a haplotype block contains only a single common SNP, the flanking common SNPs on either side of the single common SNP represent the outer common SNPs of adjacent haplotype blocks. A polymorphic form of a common SNP in a haplotype block that contains only one common SNP is not predictive of the polymorphic form of any other common SNPs.

In some instances, a haplotype pattern of multiple polymorphic forms at multiple polymorphic sites can be defined from the presence of a single polymorphic form at a single polymorphic site (i.e., a single haplotype-defining polymorphism). In other instances, the identity of more than one haplotype-defining polymorphism within a given haplotype block is required to identify the haplotype pattern that occupies that block. For example, the polymorphic form of a haplotype-defining SNP located in a haplotype block that contains multiple common SNPs can identify the haplotype pattern as one of two possible haplotype patterns and rule out two other haplotype patterns. In such an instance, at least one more haplotype-defining SNP must therefore be identified in the same haplotype block before the haplotype pattern that occupies the haplotype block can be unambiguously identified. In general, a smaller number of haplotype-defining SNPs must be analyzed to distinguish between the four most common haplotype patterns in a given haplotype block, whereas a larger number of haplotype-defining SNPs must be analyzed to distinguish between more than the four most common haplotype patterns.

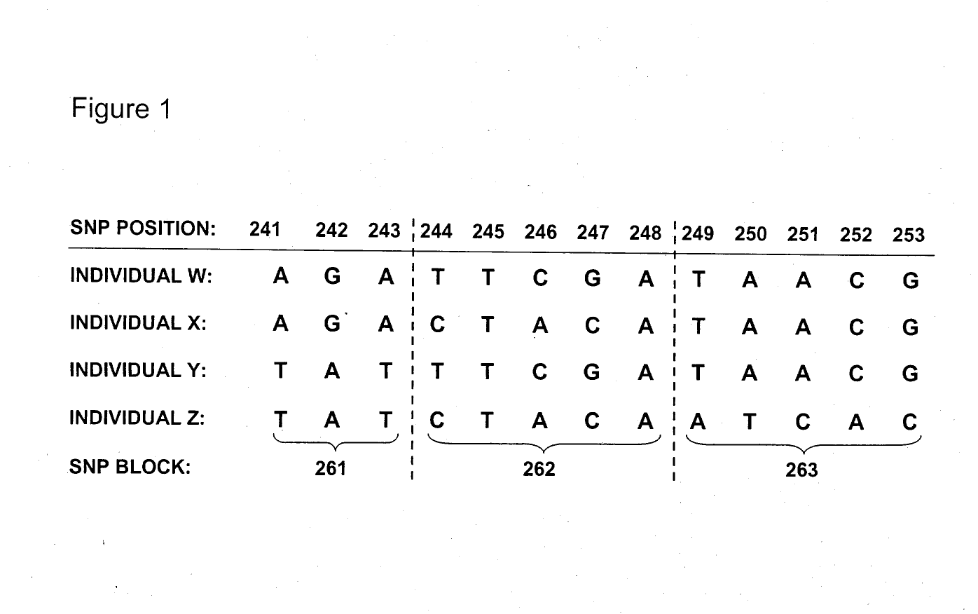

FIG. 1 provides one illustration of how SNPs occur in blocks throughout a genome. Such haplotype blocks are chromosomal regions that tend to be inherited as a unit, typically with a relatively small number of common forms. Each line in FIG. 1 represents portions of the haploid genome sequence of different individuals. Individual W has an “A” at position 241, a “G” at position 242, and an “A” at position 243. Individual X has the same bases at positions 241, 242, and 243. Conversely, individual Y has a T at positions 241 and 243, but an A at position 242. Individual Z has the same bases as individual Y at positions 241, 242, and 243. The SNPs are most commonly biallelic. Variants in block 261 tend to occur together. Similarly, the variants in block 262 tend to occur together, as do the variants in block 263. Only a few nucleotides in the haplotype blocks are shown in FIG. 1. Most nucleotides in a genome are like those at position 245 and 248, and do not vary between genomes of the same species, and hence are not considered to be polymorphic sites. This tendency of SNPs to occur together in haplotype blocks allows for a single haplotype-defining SNP or a few haplotype-defining SNPs in a haplotype block to be analyzed to identify haplotype patterns, rather than analyzing all of the SNPs in that haplotype block. For example, by identifying only the SNP at position 241, the SNPs at positions 242 and 243 can be predicted without performing an assay to identify SNPs 242 and 243. If position 241 contains an A, position 242 contains a G and position 243 contains an A. Conversely, if position 241 contains a T, positions 242 and 243 contain an A and a T, respectively. Therefore, a haplotype-defining SNP occurs at position 241.

A plurality of haplotype-defining SNPs may be analyzed in the genomes of the samples to determine which haplotype patterns are present at haplotype blocks throughout the genome, optionally at least 25,000, 100,000 or 200,000 haplotype blocks, in certain embodiments up to 1,000,000 haplotype blocks. Haplotype blocks may contain between one and ten or more haplotype-defining SNPs. The more haplotype blocks that are analyzed, the greater the chances are of identifying a haplotype pattern associated with the differential relative allelic expression pattern of a gene. Preferably substantially all haplotype blocks in a genome are analyzed. When all haplotype blocks in a genome are analyzed, essentially the entire genome of the individual is analyzed. Some haplotype blocks contain over 100 SNPs. Some haplotype blocks are over 100 kb in length. Other haplotype blocks are less than 5 kb in length. For a general explanation of determining the number of haplotype-defining SNPs that must be identified to distinguish between haplotype patterns, see Patil et al., Science 2001 Nov. 23;294(5547):1719-23.

### C. Association Methods Using Identified Haplotype Patterns

### 1. Generation of Haplotype Pattern Association Data

In some embodiments of the present invention, samples that demonstrate similar or identical differential relative allelic expression patterns for a gene form a test group. Samples that do not demonstrate a differential relative allelic expression for the same gene form the control group. Alternatively, the control group may comprise samples that demonstrate different differential relative allelic expression patterns for a gene from those of the test group. For example, one group (e.g. test group) in a study may comprise individuals that display a differential relative allelic expression pattern in which the reference allele is expressed at a higher level than the alternate allele (reference<alternate), and a second group (e.g. control group) in the study may comprise individuals that display a differential relative allelic expression pattern in which the reference allele is expressed at a lower level than the alternate allele (reference<alternate). The frequency of each haplotype pattern among samples in the test group is compared to the frequency of the same haplotype patterns among samples in the control group. Haplotype patterns that occur among samples in the test group at a statistically significantly different frequency than the frequency at which they occur among samples in the control group are associated with the differential relative allelic expression pattern for that gene. The same type of analysis can be performed for individual polymorphic forms at individual polymorphic sites. For general methods of performing association studies with a phenotypically-defined population and a control population see Kristensen, et al., “High-Throughput Methods for Detection of Genetic Variation”, BioTechniques 30(2):318-332 (2001) and Kirk, et al., “Single nucleotide polymorphism seeking long term association with complex disease”, Nucleic Acids Research 30(15): 3295-3311 (2002).

The comparison of haplotype pattern frequencies is performed for each gene for which differential relative allelic expression patterns are determined. Each sample exhibits differential relative allelic expression patterns only at a subset of the genes analyzed, and different samples are unlikely to exhibit the same differential relative allelic expression patterns for the same subset of genes. In some instances, one group in a study may comprise individuals that display a differential relative allelic expression pattern in which the reference allele is expressed at a higher level than the alternate allele (reference<alternate) for one subset of one or more genes, and a differential relative allelic expression pattern in which the reference allele is expressed at a lower level than the alternate allele (reference<alternate) for another subset of one or more genes. In these instances, association analysis is performed to identify haplotype patterns associated with both patterns.

For example, if sample 1 exhibits a differential relative allelic expression pattern of reference<alternate for gene 1, its haplotype patterns are included in the test group for analysis of gene 1. If sample 1 is heterozygous for gene 2 but does not exhibit a differential relative allelic expression pattern for gene 2, its haplotype patterns are included in the control group for analysis of gene 2. Haplotype patterns from a sample are not included in the test group or control group for analysis of a gene if the sample is homozygous at the transcribed SNP position in that gene. This is because such a sample is not capable of exhibiting or not exhibiting differential relative allelic expression patterns for the given gene because the alleles of the gene are not different. The test groups and control groups may therefore comprise a different subset of samples for the association analysis for each gene that exhibits a differential relative allelic expression pattern. The invention therefore provides methods wherein during investigation of a plurality of differentially expressed genes the same haplotype pattern data for a sample is analyzed as part of the test group for a first subset of one or more genes, as part of the control group for a second subset of one or more genes, or not analyzed for a third subset of one or more genes for which the sample is homozygous.

### 2. Mechanisms of Differential Relative Allelic Expression Pattern Modulation

Although knowledge of the mechanism of how SNPs alter expression levels of different alleles of a gene is not necessary to practice the invention, it is believed that some SNPs modify the aggregate scaffolding of proteins along a chromosome. Some SNPs alter the amino acid sequence, and therefore the activity, expression and/or affinity of proteins that bind to chromosomes. When each copy of a chromosome in a diploid cell differs in sequence at the same locus due to the presence of different haplotype patterns, there may be a slightly different aggregate scaffolding of proteins along each of the respective chromosomes that affects the expression of genes on that chromosome and/or on other chromosomes in quantifiable ways. Many characteristics of the proteins that comprise the aggregate scaffolding, such as total copy number of each protein in the cell, post-translational modification of each protein, and the ability to recruit other proteins to the chromosome, are in turn determined by the identity of SNPs located throughout the entire genome. The existence of SNPs within haplotype blocks located within and outside of coding regions of genes throughout the genome therefore creates a variable network of chromosome binding proteins and DNA sequence elements that recruit chromosome binding proteins with differential affinity based on sequence. The identity of each haplotype pattern throughout the genome therefore modulates the variable network, and this modulation manifests through the differential relative allelic expression patterns of genes.

Some genes exhibit differential relative allelic expression patterns depending on the presence or absence of certain haplotype patterns that modulate the function of the variable network. However, other pathways that may also be involved in differential relative allelic expression patterns include, but are not limited to, transcriptional regulation pathways (e.g. involving enhancer sequences), post-transcriptional modification pathways (e.g. splicing), mRNA degradation pathways, translational regulation pathways, post-translational modification pathways (e.g. phosphorylation, methylation and glycosylation), and protein degradation pathways. Because there are hundreds of thousands, perhaps millions of haplotype blocks throughout the human genome, each of which may contain one of a number of different possible haplotype patterns, an enormous number of haplotype patterns can wholly or in part cause differential relative allelic expression patterns of genes. The methods of the invention identify haplotype patterns that cause differential relative allelic expression patterns of genes. Such haplotype patterns can be associated with diseases caused by overexpression or underexpression of certain genes.

### 3. Results of Association Analysis

Several different types of associations between differential relative allelic expression patterns of a gene and specific haplotype patterns are found when a significant number of genes are analyzed. In some instances the differential relative allelic expression patterns of a gene are not associated with the presence of any particular haplotype pattern. In other instances the differential relative allelic expression patterns of a gene are associated with the presence of a single haplotype pattern. In other instances the differential relative allelic expression patterns of a gene are associated with the presence of a plurality of distinct haplotype patterns found in a single haplotype block. In other instances the differential relative allelic expression patterns of a gene are associated with the presence of a plurality of distinct haplotype patterns found in distinct haplotype blocks. In still other instances the differential relative allelic expression patterns of a gene are associated with a plurality of haplotype patterns, such that at least two of the haplotype patterns occur in the same haplotype block and at least two of the haplotype patterns occur in different haplotype blocks. A haplotype block that is associated with the differential relative allelic expression pattern of a given gene may reside on the same chromosome as the gene, or may reside on a different chromosome. In some instances, one or more haplotype patterns found to associate with differential relative allelic expression levels of a gene also associate with one or more other genes.

Haplotype patterns associating with differential relative allelic expression can occur within a transcribed region of a gene, proximal thereto, or distal thereto. If a haplotype block overlaps or is proximal to a gene and a haplotype pattern of the haplotype block is found to associate with the differential relative allelic expression of the gene, the haplotype pattern may or may not include the polymorphism within a transcribed region of the gene that was used in determining differential relative allelic expression of the gene. Polymorphisms in the associated haplotype pattern that are within or proximal to the gene may, but do not necessarily, occur within regulatory regions that affect transcription, such as promoters, enhancer regions, or introns. Polymorphisms in the associated haplotype pattern that are within or proximal to a gene may be causally associated with differential expression or may be in linkage disequilibrium with a polymorphism that is causally associated with differentially expression. Distal associated haplotype patterns can occur on the same chromosome as the gene that is differentially expressed or on any other chromosome. Distal haplotype patterns usually occur outside regulatory regions of a differentially expressed gene and may be associated with differential relative allelic expression through trans effects.

Haplotype patterns associated with differential expression can contain polymorphic forms at one or multiple polymorphic sites. For haplotype patterns containing multiple polymorphic forms at multiple polymorphic sites, one, several, all or none of the polymorphic forms may be causally associated with differential expression (that is, may be “functional polymorphisms”). For example, for some such haplotype patterns, a single polymorphic form is causally associated with differential expression and polymorphic forms at other polymorphic sites in the haplotype pattern are in linkage disequilibrium with it. In other such haplotype patterns, multiple polymorphic forms at multiple polymorphic sites are causally associated with the differential expression. In some instances, a polymorphic form at a polymorphic site, e.g., an SNP, not directly involved in differential expression (i.e., not causally associated) is used as a marker to identify another polymorphic form that is directly involved in differential expression (i.e., causally associated). In some instances, multiple haplotype patterns that occupy different haplotype blocks are associated with a differential relative allelic expression pattern of a gene. Some of these associated haplotype patterns cumulatively associate with extent of differential relative allelic expression patterns of genes (i.e., each haplotype pattern associates independently with differential allelic expression but the extent of association is greater in the simultaneous presence of both haplotype patterns than either alone). For example, extent of association can be measured by a Chi squared value in which case the Chi squared value for association of the haplotype patterns in combination is greater than that for each haplotype pattern individual. The combination may or may not be synergistic. Other haplotype patterns do not associate independently but only in combinations of two or more haplotype patterns. Distal haplotype patterns associating with differential expression usually do so in combination with a haplotype pattern within or proximal to a gene. In some methods, associations between haplotype patterns and differential relative allelic expression patterns are first performed for haplotype blocks within or proximal to the transcribed regions of a gene. Once such a haplotype pattern associated with differential relative allelic expression of the gene has been identified, additional association analyses are performed for haplotype blocks at more distal locations with respect to the differentially expressed gene. In these additional association analyses, samples may be classified into groups depending both on the presence or absence of differential relative allelic expression patterns and the presence or absence of the proximal haplotype pattern that is associated with the differential relative allelic expression pattern. These methods identify additional haplotype patterns located distal to the gene that are associated with the differential relative allelic expression pattern. The association of the additional haplotype pattern(s) may or may not be dependent on presence of the proximal haplotype pattern found to be associated with differential relative allelic expression pattern.

Some differential relative allelic expression patterns of a gene may be identified that are associated with a first haplotype pattern at a statistically significant level (p<0.05) in some individuals and not others. In such instances, the differential expression pattern may associate with a second and possibly more haplotype patterns in the genome that are also necessary for generating the differential relative allelic expression pattern of the gene. A second haplotype pattern associated with the differential relative allelic expression pattern can be identified by performing an association study in which the control group is a group of individuals that do not display the differential relative allelic expression pattern for the gene and the test group is a group of individuals that do display the differential relative allelic expression pattern. Both the test and control groups contain the first identified haplotype pattern and are heterozygous for the differentially expressed gene. A second haplotype pattern that is associated at a statistically significant level with the test group but not the control group may be associated with the differential relative allelic expression pattern. There may be a plurality of haplotype patterns that are associated with the differential relative allelic expression pattern, all of which are necessary but none of which is by itself sufficient to cause the differential relative allelic expression pattern. When the differential relative allelic expression pattern is associated with a plurality of haplotype patterns, the associated haplotype patterns may be located in the same haplotype block, or in different haplotype blocks. When the associated haplotype patterns are located in different haplotype blocks, they may be located on the same chromosome or on different chromosomes. Some associated haplotype patterns may be located in haplotype blocks that overlap or partially overlap the gene. Other associated haplotype patterns are located in haplotype blocks that do not overlap the gene and may be located on the same or a different chromosome than the gene.

Alternatively from the above, it may be found that a differential relative allelic expression pattern is associated with a plurality of haplotype patterns, wherein zero, one, or more haplotype patterns are individually capable of generating the differential relative allelic expression pattern. In other words, in some instances it may be the case that each associated haplotype pattern exerts a cumulative effect on generating the differential relative allelic expression pattern, and that the presence of only one haplotype pattern in the cell is not enough to generate the pattern. In such instances it may be found that the more associated haplotype patterns that are present within a cell, the greater the difference in expression levels between the two alleles. In these instances some associated haplotype patterns exert a cumulative effect on the magnitude of the difference in expression between the alleles rather than an “all or none” effect on whether there is or is not a difference in expression between the two alleles. Further, these cumulative effects may be complementary or antagonistic; i.e., some combinations may cause a greater differential in allelic expression [e.g. (ref>alt)+(ref

>alt)=(ref>>alt)] while others may lessen the observed difference in allelic expression [e.g. (ref>>alt)+(ref<alt)=(ref>alt)].

Other methods of investigating haplotype patterns that are associated with differential relative allelic expression patterns may be employed. For example, in some instances it is found that the magnitude of the difference in expression levels between two alleles varies between individuals that all exhibit the same differential relative allelic expression pattern for a gene, e.g., reference>alternate. Haplotype patterns that are responsible for the difference in magnitude of the differential relative allelic expression pattern are identified by performing an association study in which a first group of individuals displays a first ratio of expression levels between the two alleles and a second group of individuals displays a second, distinct ratio of expression levels between the two alleles. Haplotype patterns that are present in the second group at a statistically significantly higher frequency than in the first group are associated with the difference in magnitude of the differential relative allelic expression levels of the gene between the second and first groups, as are those present in the first but not the second group. This example demonstrates that a plurality of samples for which both haplotype patterns and expression levels of heterozygous genes have been identified may be grouped in a variety of ways for the purpose of stratifying the samples to identify haplotype patterns that independently exert different effects on gene expression.

### VI. Uses of Identified Genomic Sequences that are Associated with Differential Relative Allelic Expression Patterns

In some methods, haplotype-defining SNPs or haplotype patterns that are associated with differential relative allelic expression patterns for a given gene are further analyzed for association with certain phenotypes, such as the occurrence of a particular disease state, the resistance to a particular disease state, the occurrence of an adverse reaction to a drug, the occurrence of an efficacious reaction to a drug, the occurrence of no reaction to a drug, and other phenotypes. In some methods provided, haplotype blocks that contain haplotype patterns that are associated with a differential relative allelic expression pattern for a given gene are further analyzed to identify genes that are located partially or completely within the haplotype blocks, and that contribute to or cause the differential relative allelic expression pattern.

### A. Disease Targets

Once a haplotype pattern or multiple haplotype patterns are associated with a differential relative allelic expression pattern of a gene, the gene(s) or regulatory elements located partially or completely within or proximate to the haplotype block or blocks are identified (hereafter, “the identified gene”). Identification of genes located partially or completely within or proximate to a haplotype block that contains an associated haplotype pattern is facilitated by knowledge of the complete human genome sequence. Genes located in a particular region of the human genome can be identified through resources such as the National Center for Biotechnology Information located at http://www.ncbi.nlm.nih.gov/genome/guide/human. Genes can be identified by scanning the sequence within or proximate (e.g., within 10 kb of the outermost polymorphic sites within the block) to haplotype block(s) correlated with differential allelic expression for open reading frames. Expression of such genes can be tested by hybridization of probes based on the gene sequence to mRNA prepared from a tissue of interest.

In some instances, the increased expression of a gene that exhibits differential relative allelic expression patterns is known to be associated with particular disease state. For example, a common SNP in the coding region of the angiotensinogen gene that changes a methionine residue to a threonine residue at position 235 in the amino acid sequence has been found to occur at a higher frequency in individuals with essential hypertension, a common disease affecting millions of individuals in the United States alone, than in individuals with normal blood pressure. Jeunemaitre et al., Cell 1992 Oct. 2;71(1):169-80. Furthermore, the allele containing a threonine at position 235 is expressed at a higher level than the allele containing methionine at position 235. Inoue et al., J Clin Invest 1997 Apr. 1;99(7):1786-97. No mechanism for this differential relative allelic expression has to date been elucidated, however it is known that increasing the expression of the angiotensinogen gene results in an increase in blood pressure. Kim et al., Proc Natl Acad Sci USA 1995 Mar. 28;92(7):2735-9. The invention provides methods for identifying haplotype patterns that are associated with the differential relative allelic expression of disease-causing alleles of genes such as angiotensinogen. Haplotype patterns associated with the differential relative allelic expression pattern of genes such as angiotensinogen can in some instances identify not only expressed genes that can investigated for treating the disease state, but the associated haplotype pattern can also provide information about the biological basis of the differential relative allelic expression pattern and/or the disease. The genes or regulatory elements located partially or completely within or proximate to the associated haplotype block (“the identified genes”) are therefore investigated as therapeutic targets for the treatment of disease states such as essential hypertension.

To determine how the genes or proteins encoded by the identified gene may be manipulated to treat disease, the sequence of the identified gene, including flanking promoter regions and coding regions, can be altered in various ways to generate targeted changes in expression level or changes in the sequence of the encoded protein. The sequence changes can be substitutions, insertions, translocations or deletions. Deletions can include large changes, such as deletions of an entire domain or exon. Examples of protocols for site specific mutagenesis can be found in, e.g., Gustin, et al., Biotechniques 14:22 (1993) and Sambrook, et al., Molecular Cloning: A Laboratory Manual (Cold Spring Harbor Press) pp. 15.3-15.108 (1989). Such altered genes can be used to study structure/function relationships of the protein product, or to change the properties of the protein that affect its function or regulation.

The identified gene can be employed for producing all or portions of the resulting polypeptide. To express a protein product, an expression cassette incorporating the identified gene can be employed. The expression cassette or vector generally provides a transcriptional initiation region, which can be inducible or constitutive. The coding region is operably linked under the transcriptional control of the transcriptional initiation region, a translational initiation region, and a transcriptional and translational termination region. These control regions can be native to the identified gene, or can be derived from exogenous sources.

The identified gene can be expressed in cells that also contain the differentially expressed alleles of the gene (“gene X”) that exhibits differential relative allelic expression patterns. The sequence of the identified gene can be manipulated in various ways to determine the mechanism(s) through which it exerts a differential effect on the two alleles of gene X. For example, the identified gene may be expressed in diploid cells containing both alleles of gene X wherein the cDNA encoding the identified gene contains variants from the associated haplotype pattern and the differential relative allelic expression patterns of gene X are assayed. The identified gene is also expressed wherein the cDNA encoding the identified gene contains variants from other non-associated haplotype patterns. This experimental method can elucidate whether the amino acid sequence of the identified gene is responsible or partially responsible for the differential relative allelic expression patterns of gene X. Differential relative allelic expression patterns can also be investigated in cells exposed to molecules that inhibit or enhance the function of the identified gene.

The protein encoded by the identified gene can be used for the production of antibodies. Short fragments of the protein induce the production of antibodies specific for the particular polypeptide (monoclonal antibodies), and larger fragments or the entire protein allow for the production of antibodies over the length of the polypeptide (polyclonal antibodies). Antibodies are prepared in accordance with conventional ways in which the expressed polypeptide or protein is used as an immunogen, by itself or conjugated to known immunogenic carriers, e.g. KLH, pre-S HBsAg, or other viral or eukaryotic proteins. For further description, see for example Monoclonal Antibodies: A Laboratory Manual, Harlow and Lane, eds. (Cold Spring Harbor Laboratories, Cold Spring Harbor, N.Y.) (1988).

The identified genes, gene fragments, or the encoded protein or protein fragments can be useful in gene therapy to treat degenerative and other disorders. For example, expression vectors can be used to introduce the identified gene into a cell. Such vectors generally have convenient restriction sites located near the promoter sequence to provide for the insertion of nucleic acid sequences in a recipient genome. Transcription cassettes can be prepared comprising a transcription initiation region, the target gene or fragment thereof, and a transcriptional termination region. The transcription cassettes can be introduced into a variety of vectors such as plasmids, retroviruses such as lentivirus and adenovirus, in which the vectors are able to be transiently or stably maintained in the cells. The gene or protein product can be introduced directly into tissues or host cells by any number of routes, including viral infection, microinjection, or fusion of vesicles.

Antisense molecules may be used to downregulate expression of the identified gene in cells. The antisense reagent may be antisense oligonucleotides, particularly synthetic antisense oligonucleotides having chemical modifications, or nucleic acid constructs that express such antisense molecules as RNA. A combination of antisense molecules can be administered, in which a combination can comprise multiple sequences. As an alternative to antisense inhibitors, catalytic nucleic acid compounds such as ribozymes and antisense conjugates can be used to inhibit gene expression. Another alternative to antisense molecules is an RNAi (RNA interference) construct. Expression of RNAi constructs generate double stranded RNA molecules that inhibit the expression of genes that share sequence identity with the RNAi molecule. For example, see Cioca et al., Cancer Gene Ther 2003 Feb;10(2):125-33. Antisense or RNAi molecules may be employed to downregulate the expression of an identified gene that is associated with the differential relative allelic expression patterns.

Genetic function can be investigated with non-mammalian models, particularly using those organisms that are biologically and genetically well-characterized, such as C. elegans, M. musculus, D. melanogaster and S. cerevisiae. The identified gene sequences can be used to knock out corresponding gene function or to complement defined genetic lesions to determine the physiological and biochemical pathways involved in protein function. Drug screening can be performed in combination with complementation or knock out studies, e.g., to study progression of degenerative disease, to test therapies, or for drug discovery.

Protein molecules encoded by identified genes can be assayed to investigate structure/function parameters. For example, by providing for the production of large amounts of a protein product of an identified gene, one can identify ligands or substrates that bind to, modulate or mimic the action of that protein product. Drug screening identifies agents that provide, e.g., a replacement or enhancement for protein function in affected cells, or for agents that modulate or negate protein or mRNA function. Some agents identified by drug screening interact (e.g., specifically bind) with protein or mRNA. Some agents interact with an entity such as a ligand, receptor, or transcription factor that itself interacts with protein or mRNA. Some agents alter the differential relative allelic expression pattern by inhibiting or stimulating, either directly or indirectly, the transcription of an expressed gene. Some agents later the differential relative allelic expression pattern by inhibiting or stimulating, either directly or indirectly, the translation of the mRNA encoded by the expressed gene.

Candidate agents encompass numerous chemical classes, though typically they are organic molecules or complexes, preferably small organic compounds, having a molecular weight of more than 50 and less than about 2,500 daltons, and can be obtained from a wide variety of sources including libraries of synthetic or natural compounds.

Where the screening assay is a binding assay, one or more of the molecules can be coupled to a label. The label can directly or indirectly provide a detectable signal. Various labels include radioisotopes, fluorescers, chemiluminescers, enzymes, and specific binding molecules, particles such as magnetic particles. Specific binding molecules include pairs such as biotin and streptavidin, and digoxin and antidigoxin. For the specific binding members, the complementary member is normally labeled with a molecule that provides for detection, in accordance with known procedures.

Any of the preceding methods can be employed for the purpose of investigating the function of identified genes. In some instances, as previously mentioned, a single haplotype pattern is associated with the differential relative allelic expression patterns of more than one gene. Some methods provided herein are directed toward the investigation of single haplotype patterns associated with the differential relative allelic expression patterns of a plurality of genes. When a gene that is located partially or completely within or proximate to a haplotype block that contains an associated haplotype pattern is itself modulated through techniques described herein, such as RNAi, the differential relative allelic expression patterns of a plurality of genes can therefore be altered through the modulation of a single identified gene. Some methods provided are therefore directed to the modulation of plieotropic effects, wherein the plieotropic effects comprise the differential relative allelic expression patterns of a plurality of genes associated with a single haplotype pattern.

### B. Clinical Trials

Haplotype patterns found to be associated with a differential relative allelic expression pattern may also be used to determine drug responsiveness in a clinical trial of a pharmaceutical composition. For example, when a gene is known to play a role in the metabolism of a particular drug, the gene can be assayed for differential relative allelic expression patterns. Haplotype patterns that are associated with a differential relative allelic expression pattern of such a gene are then identified. The presence or absence of haplotype patterns associated with a differential relative allelic expression pattern are then analyzed for association with the response or lack thereof of a patient to the drug. Generally a patient A responds at a level indicating efficacy of the drug, B responds but at a level not indicating efficacy of the drug, C does not respond at all to the drug, or D has an adverse reaction to the drug. Haplotype patterns that are associated with a differential relative allelic expression pattern are analyzed for association with one of these four outcomes. In some instances it is found that the associated haplotype pattern is associated with a particular outcome. It can also be found that different haplotype patterns at the same haplotype block are associated with different outcomes. In other instances there is no association. In instances in which a haplotype pattern that is associated with a differential relative allelic expression pattern also is associated with an adverse reaction to a drug, genes identified partially or completely within or proximate to the haplotype block that contains the associated haplotype pattern are investigated as targets for the elimination of the adverse response using methods previously described herein.

The methods provided can identify haplotype patterns that, when present in an individual, are associated with an adverse reaction to a certain drug or a certain class of drugs. In some instances these adverse reactions may be averted through modulation of genes located in haplotype blocks that contain associated haplotype patterns. In other instances, in clinical trials, patients with certain haplotype patterns are given different drugs or different doses of the drug to avoid these adverse effects. In some instances the dose and identity of a drug is determined by which haplotype patterns occur in a patient in a clinical trial.

The methods of the present invention may also be used for diagnostics, such that the presence or absence of a phenotypic trait is determined by the presence or absence of a haplotype pattern that is associated with a differential relative allelic expression pattern. For example, the methods of the present invention may be used to predict the risk of an individual for developing a disease, diagnose an individual who already has the disease, or to choose a treatment or preventative regimen with the highest efficacy and fewest side-effects. For example, certain haplotype patterns discovered to be associated with a differential relative allelic expression pattern of a gene can be associated with genetically-inherited diseases that are associated with the increased or decreased expression of the gene. In such instances the patient is diagnosed by the detection of the associated haplotype pattern. The methods of the present invention can also be used on organisms aside from humans.

Various embodiments and modifications can be made to the invention disclosed in this application without departing from the scope and spirit of the invention. Unless otherwise apparent from the context any embodiment, feature or element of the invention can be used in combination with any other. All patent filings and publications mentioned herein are incorporated by reference for all purposes to the same extent as if each were so individually denoted.

### EXAMPLE 1

Materials and Methods

DNA and RNA isolation:

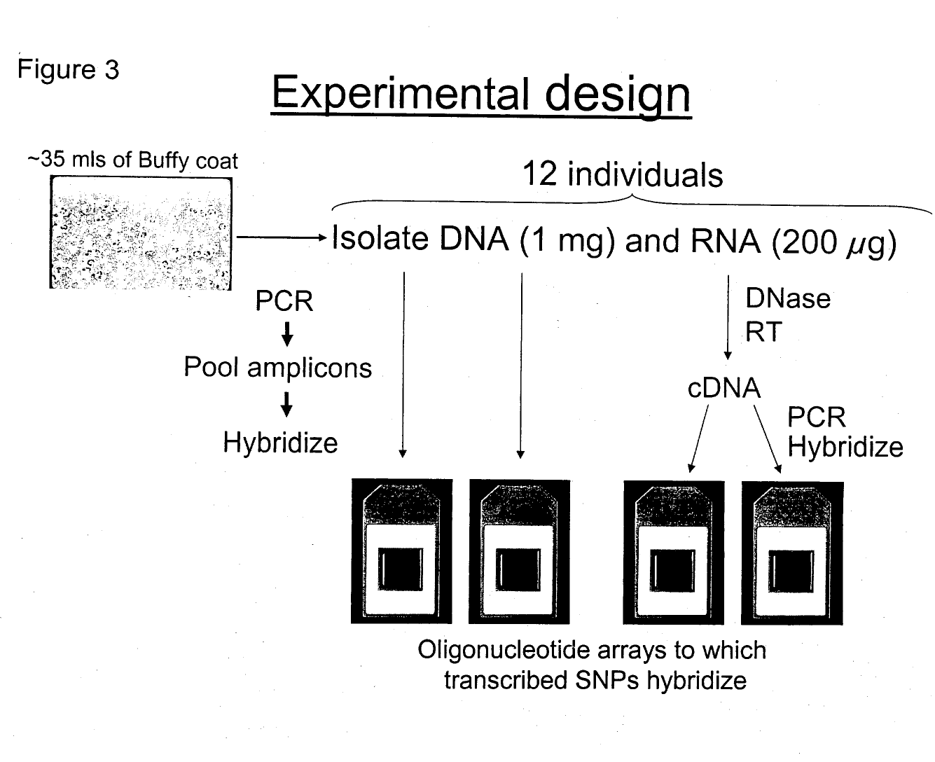

12 buffy-coats (white blood cells-enriched blood samples, 35-37 ml) were obtained from the Stanford blood center (Palo Alto, Calif.) and white blood cells were isolated by centrifugation in Ficoll density medium (Amersham Pharmacia) (see FIG. 3). The cells were then resuspended in Trizol Reagent (Invitrogen). RNA and DNA were purified in the same procedure according to manufacture's instruction. Typical yield of each sample was 200ug-400ug for RNA and ˜1 mg for DNA. Before amplification, RNA was treated with DNase I, purified again by phenol-chloroform extraction and ethanol precipitation and then subjected to reverse transcription to produce cDNA.

Short-Range PCR Reaction:

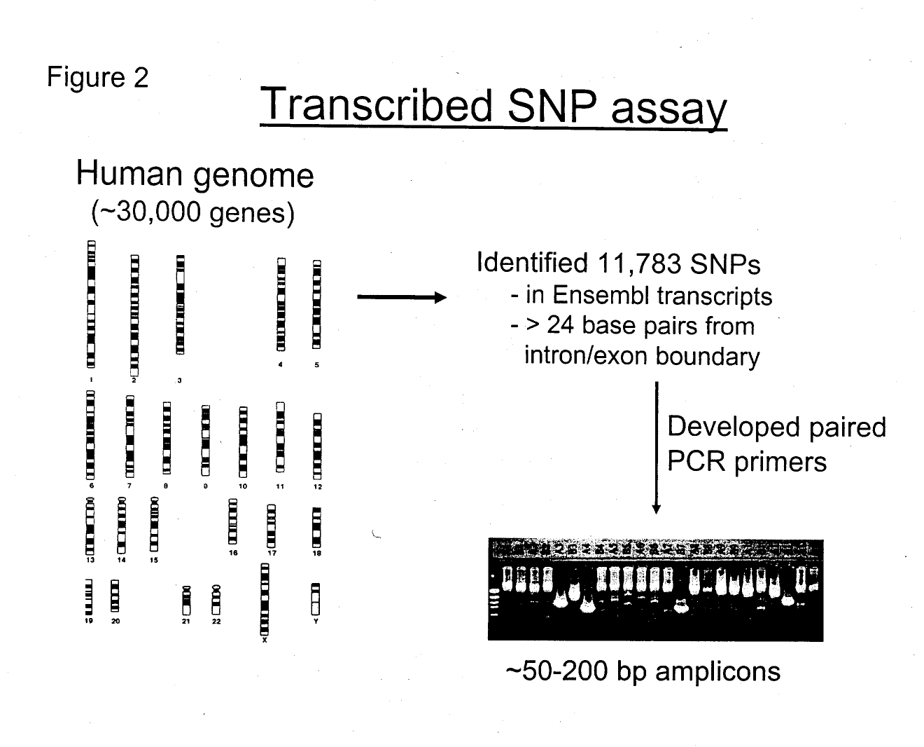

Primer selection for short-range PCR was performed as shown in FIG. 2, and essentially as described in U.S. patent application Ser. No. 10/341,832, filed Jan. 14, 2003, entitled “Apparatus and Methods for Selecting PCR Primer Pairs.” A modification of the methods described in U.S. patent application Ser. No. 10/341,832 that was used in this embodiment of the present invention is that prior to applying the Oligo primer-picking program (Molecular Biology Insights, Inc., Cascade, Colo., incorporated herein by reference), all genomic regions except those that correspond to exons were masked out of the SNP-flanking sequence. Thus, only exonic SNP-flanking sequences were used to design the short-range primers for this embodiment of the present invention. The exons were identified by aligning mRNA transcripts against the human genome. The alignment may be accomplished using any available search tool that can align nucleic acid sequences against the human genome such as, for example, BLAT (http://genome.ucsc.edu/cgi-bin/hgBlat?command=start), BLAST (http://www.ncbi.nlm.nih.gov/genome/seq/page.cgi?F=HsBlast.html&&ORG=Hs), and SSAHA (Http://www.ensembl.org/Homo_sapiens/ssahaview). Transcript sequences are also publicly available from a variety of online databases such as, for example, Ensemble (http://www.ensembl.org/) and Refseq (http.://www.ncbi.nlm.nih.gov/RefSeq/). Further, the following ranges of values were found to be suitable for short range primers for use in a PCR for amplifying SNP-containing segments of DNA for use in the present invention: 20 to 65% for % GC, and 17 to 22 nucleotides for primer length.

PCR reactions were performed in a 384-well-plate fortnat. The final concentration was 1×PCR buffer, 2.75 mM MgCl₂, 200 μM dNTP, 0.4 μM each primer, and 0.3Unit of AmpliTaq Gold DNA polymerase (Applied Biosystems). DNA or cDNA template is 2 μg for each plate. Touch down PCR was run at 95° C. for 5 min, followed by 10 cycles of 30 sec at 95° C., 30 sec at 60° C. with-0.5° C. for each cycle and 10 sec at 72° C., followed by 40 cycles of 10 sec at 95° C., 30 sec at 55° C. and 30 sec at 72° C. Quality control of PCR reactions was tested by gel electrophoresis of reactions in the first row of each 384-well-plate.

Pooling and Purification:

PCR products from the same sample and the same chip design were pooled together. 10 ml of each pool was concentrated and purified through Centricon Column (Millipore). Concentration was measured by spectrophotometer.

Labeling and Hybridization to Chips:

5 μg of each pool was labeled with Biotin ddUTP/biotin-dUTP in total volume of 37 μl in final concentration of 1XOne-Phor-All buffer, 13.5 μM Biotin ddUTP/Biotin dUTP and 0.5 unit of Terminal Transferase (Roche). Various amounts of the labeling reaction were removed to mix with hybridization buffer based on sample type and chip design. The hybridization mix was then denatured and incubated with corresponding chips for 16-18 hours at 50° C. The chips were then washed by 6×SSPE and first stained with 2.5 μg/ml Streptavidin for 15 min, and second stained with 1.25μg/ml anti-Streptavidin antibodies for 15 min, followed by third staining with Streptavidin-Cychrome for 15 min. Between each staining, chips were washed with 6×SSPE in fluid station. Finally, chips were incubated with 0.2×SSPE for 30 min and filled with 6×SSPE for scanning.

Real-Time PCR Experiment:

Real-time PCR experiments were done based on Methods from Germer, et al. (Genome Research 1999 10:258). To determine allele frequency in RNA samples, 20ng cDNA was used instead of genomic DNA

Computational Methods for Analyzing Data:

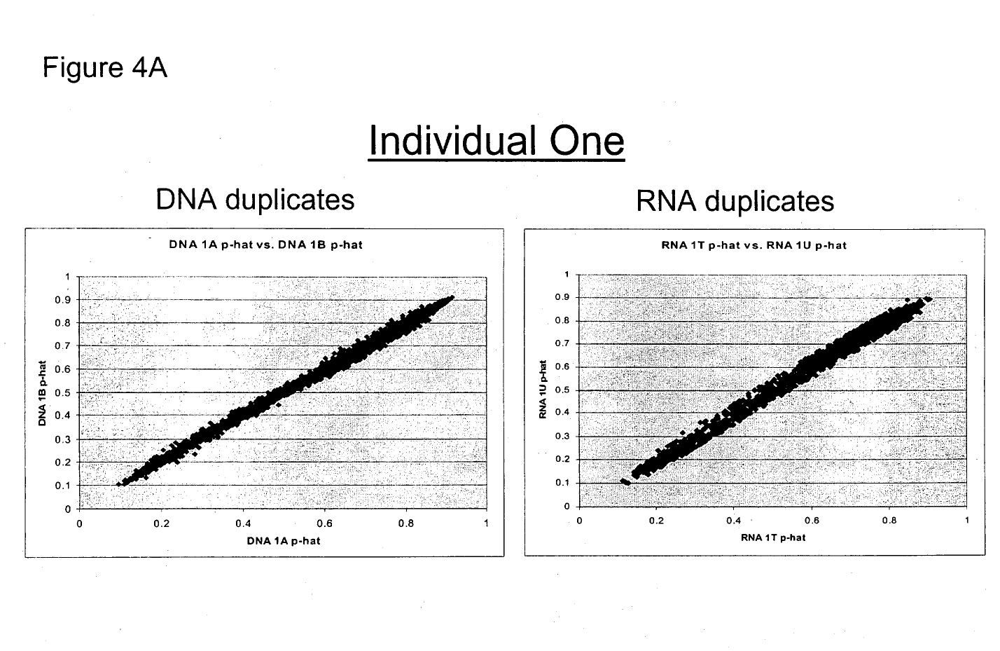

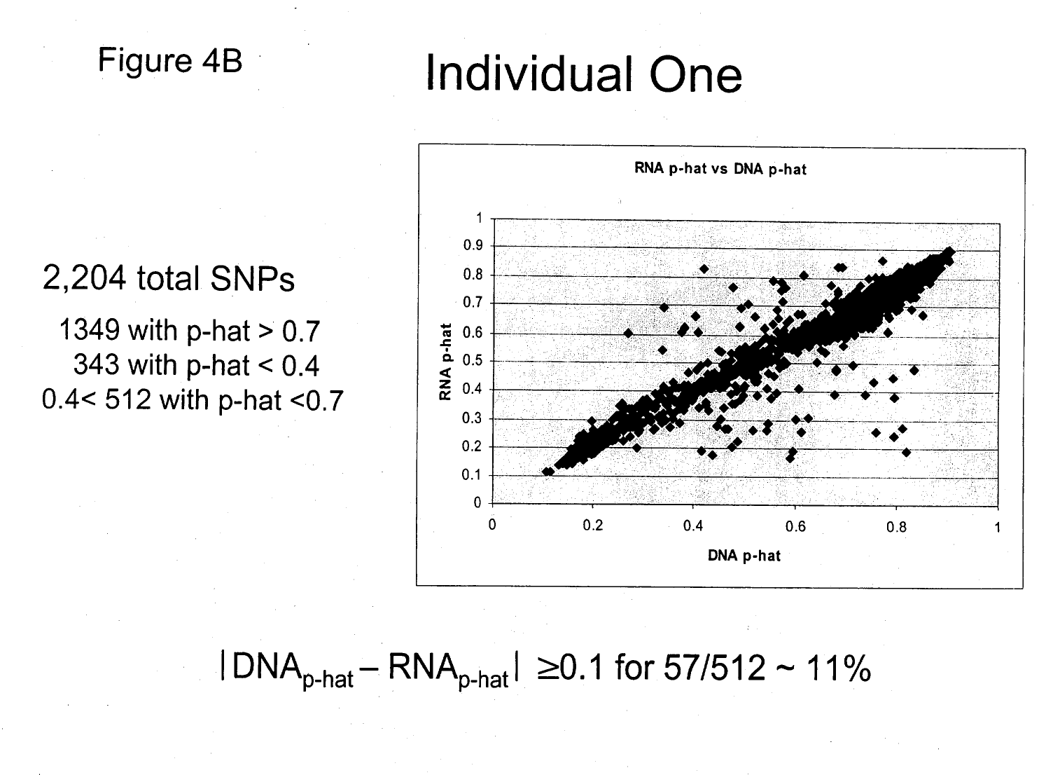

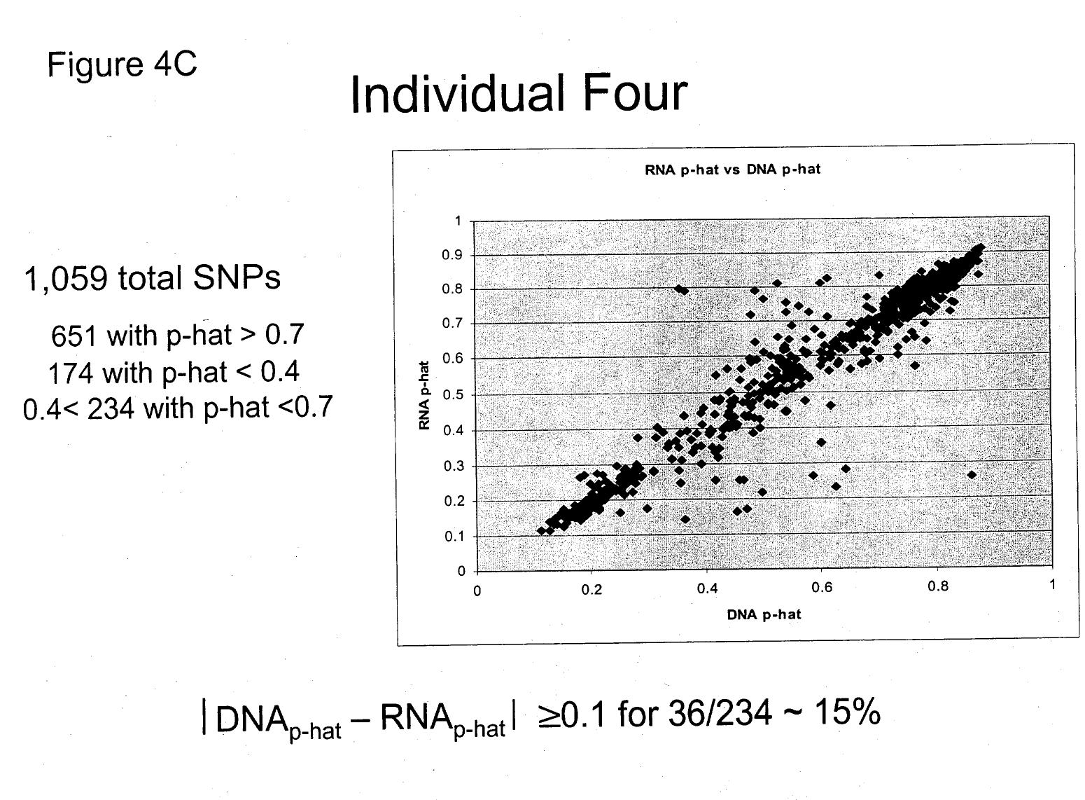

FIG. 4A is an illustrative example in which only SNPs with a p-hat difference

<0.05 between duplicates were plotted. These same SNPs were used in subsequent analyses shown in FIGS. 4B and 4C. Of course, a p-hat difference of <0.05 is not required for the present invention; other p-hat difference values may also be used to choose SNPs for subsequent analysis. FIG. 4B illustrates an experiment in which numerous genes were determined to be both heterozygous and differentially expressed between each allele. Each data point that is not on the horizontal DNA p-hat=RNA p-hat line represents a gene in Individual One that is both heterozygous and differentially expressed between the two alleles.

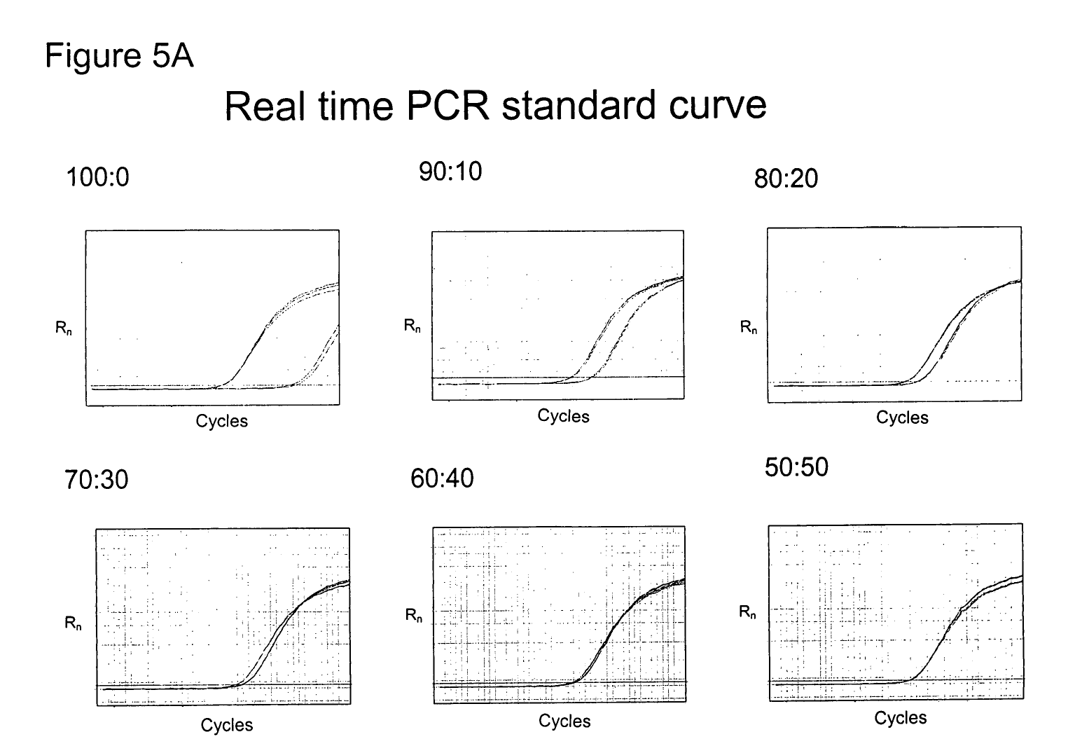

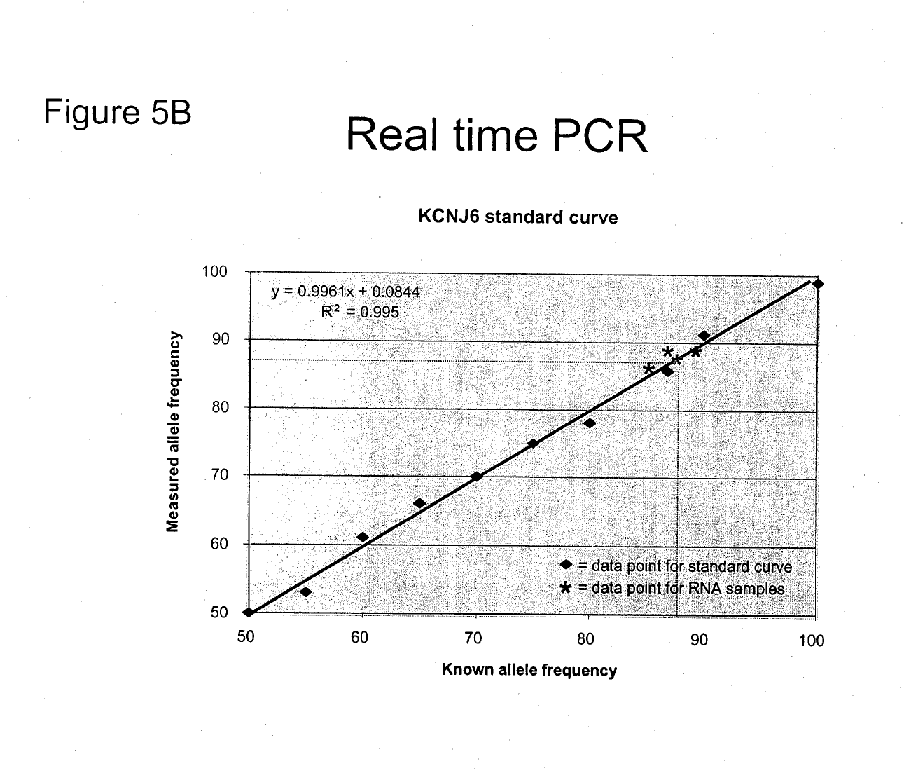

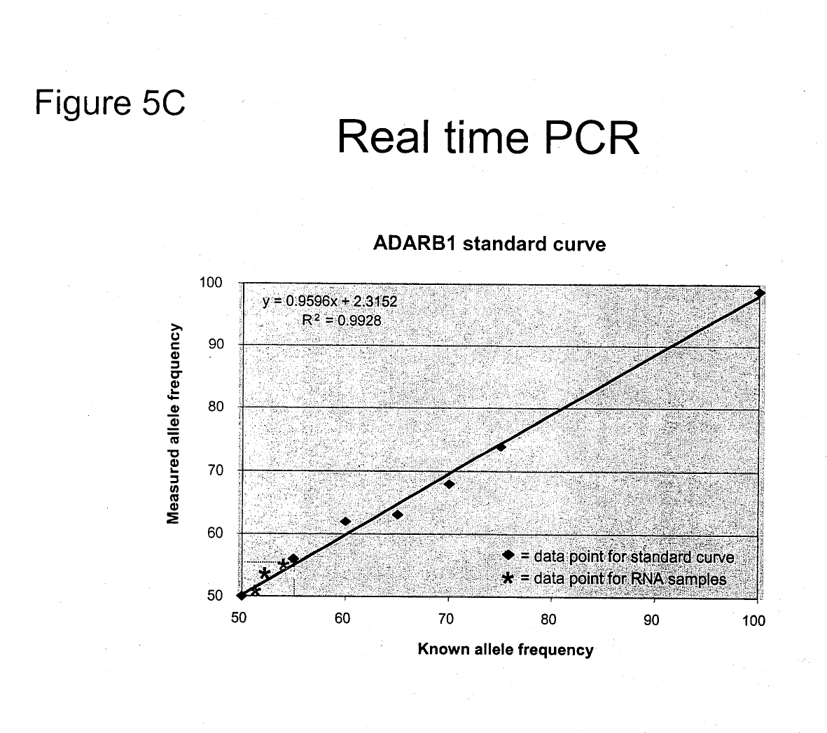

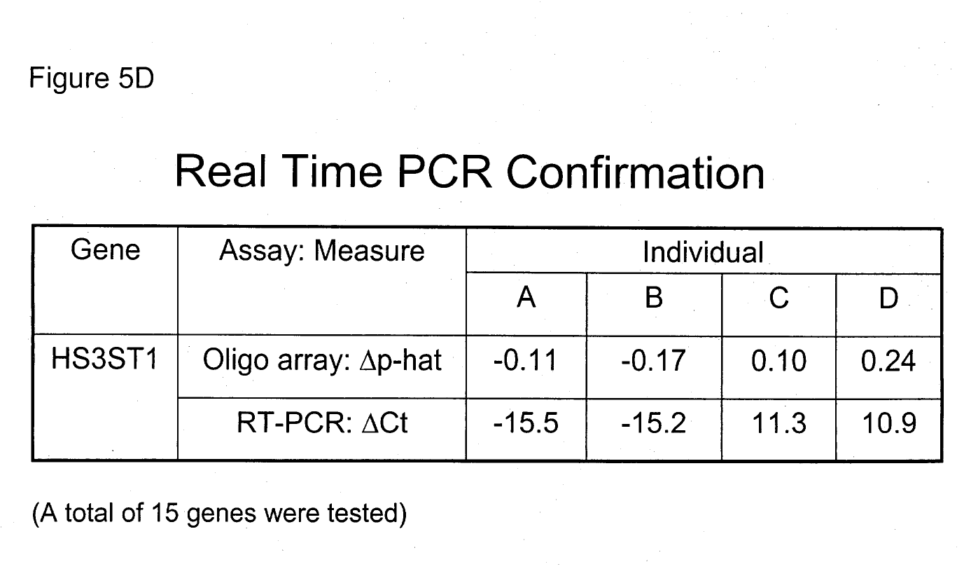

For example, in FIG. 4B each data point represents the reference allele of a particular transcribed SNP in a gene. Most of the transcribed SNPs that are heterozygous in Individual One are represented by data points that fall between approximately 0.3 and 0.7 on the DNA p-hat axis. Data points that have an RNA p-hat value of within approximately 0.1 of the DNA p-hat value represent transcribed SNPs that are encoded by reference alleles that are expressed at approximately the same level as the alternate allele for that transcribed SNP. Data points that fall between 0.4 and 0.7 on the DNA p-hat axis and have an RNA p-hat value that differs by 0.1 or more from the DNA p-hat value represent transcribed SNPs that are encoded by reference alleles that are expressed at different levels from the alternate allele and therefore indicate differential relative allelic expression patterns. FIG. 4C represents the same analysis as that depicted in FIG. 4B performed with cells from Individual Four. FIGS. 5A-D illustrate the verification of data from array hybridization by real-time PCR.

FIG. 5A illustrates that allele frequency can be calculated by real-time PCR. DNA samples from one homozygote of the reference allele and one homozygote of the alternate allele were pooled at different ratios to achieve “known” allele frequencies in the samples of 100%, 90%, 80%, 70%, 60% and 50%; the allele frequency in each sample was then measured by real-time PCR to determine the standard curve for each allele frequency. FIG. 5B illustrates allele frequencies from RNA samples from a KCNJ6 gene heterozygote measured by real-time PCR (asterisks) plotted against a standard curve generated by the data in FIG. 5A (diamonds). About 87% of the expressed RNA contains one of the two alleles present in the heterozygote, indicating that the alleles are differentially expressed. FIG. 5C illustrates that genes that do not display differential expression patterns between two alleles, such as the ADARB 1 gene, can also be detected by real-time PCR. FIG. 5D illustrates that a gene, HS3ST1, that demonstrates a differential relative allelic expression pattern based on an array data analysis also demonstrates a differential relative allelic expression pattern when analyzed with real-time PCR analysis. The same allele consistently exhibits the higher expression, regardless of the assay used, as shown by the consistency of the sign (both positive or negative) of the Δp-hat and ACt measurements. Although not shown in FIG. 5D, a total of 14 additional genes were tested and the results were consistent with those of the HS3ST1 gene.

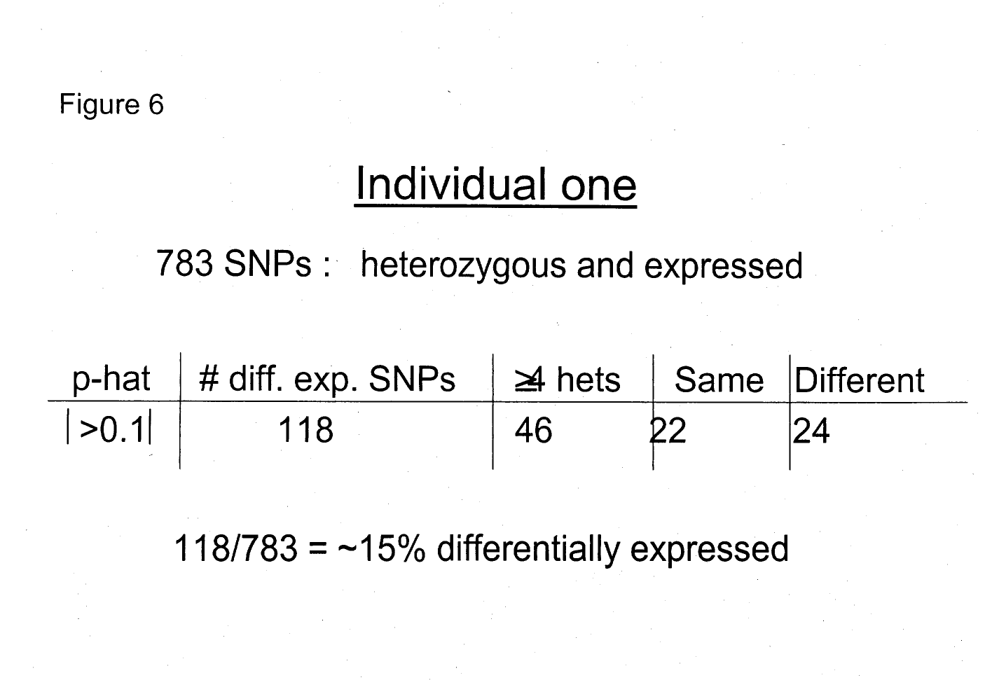

FIG. 6 illustrates that for Individual One, 783 SNPs are heterozygous and expressed. Among these SNPs, 15% have a Δp-hat between DNA and RNA>0.1, and 46 of these differentially expressed SNPs are also differentially expressed in more than 3 other heterozygous samples. For 22 of these differentially expressed SNPs, the same allele was consistently expressed at a higher level, whereas for 24 of these differentially expressed SNPs, the allele that was expressed at a higher level was different between individuals.

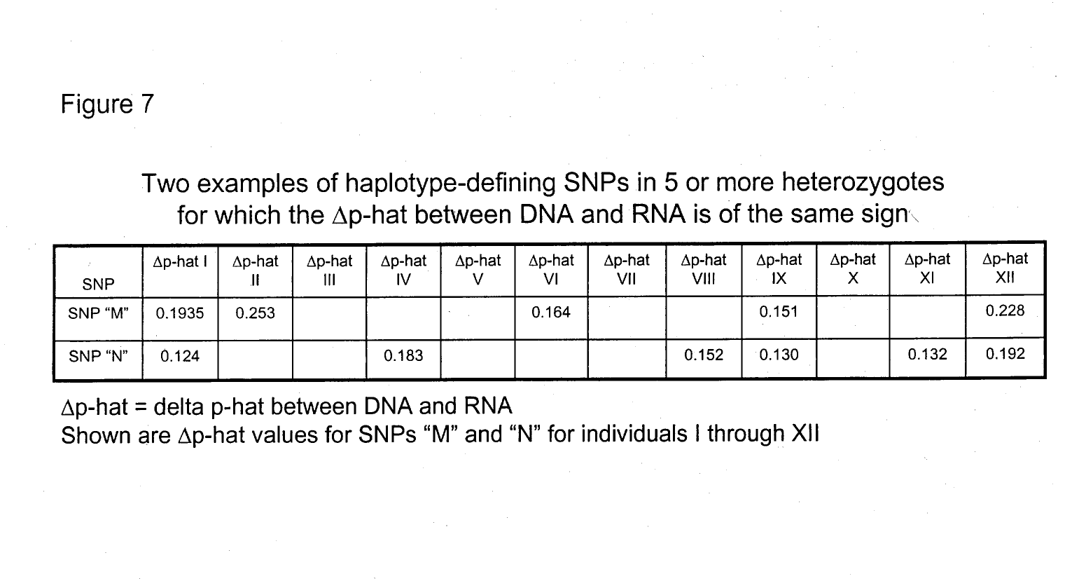

FIG. 7 illustrates two examples of haplotype defining SNPs in which 5 or more heterozygotes demonstrate similar differential relative allelic expression patterns such that the same allele is consistently expressed at a higher level.

What is claimed is:

1. A method of characterizing a gene, comprising
(a) determining a differential relative allelic expression pattern of at least two alleles of said gene from samples containing diploid cells from a plurality of individuals of the same species, wherein said cells are heterozygous for said gene;
(b) determining whether the differential relative allelic expression pattern of said gene is associated with the presence of a haplotype pattern of one or more polymorphic forms at polymorphic sites in a haplotype block, provided that if the haplotype block has only a single polymorphic site, the polymorphic site is outside the transcribed region of said gene and regulatory regions that control the transcription thereof.

2. The method of claim 1, wherein the haplotype pattern of polymorphic forms is determined by detecting a polymorphic form at a haplotype-defining polymorphic site within the haplotype block.

3. The method of claim 1, wherein the haplotype pattern of polymorphic forms is determined by detecting a plurality of polymorphic forms at a plurality of polymorphic sites within the haplotype block.

4. The method of claim 1, wherein the polymorphic sites are SNPs.

5. The method of claim 1, wherein the individuals are humans.

6. The method of claim 1, wherein the differential relative allelic expression pattern is determined from a plurality of diploid cells obtained directly from a mammalian organism.

7. The method of claim 1, the diploid cells are cultured before step (a) is performed.

8. The method of claim 1, wherein the haplotype block comprises at least ten polymorphic sites.

9. The method of claim 1, wherein the haplotype block comprises between one and ten polymorphic sites.

10. The method of claim 1, wherein the haplotype block comprises only one polymorphic site.

11. The method of claim 1, wherein the haplotype block is on a different chromosome than the gene.

12. The method of claim 1, wherein the haplotype block is on the same chromosome as the gene.

13. The method of claim 12, wherein all polymorphic sites in the haplotype block are located at least 10 kb away from said gene.

14. The method of claim 12, wherein at least one of the polymorphic sites in the haplotype block is not located within promoter, enhancer, or intronic sequences of the gene.

15. The method of claim 12, wherein at least one polymorphic site of the haplotype block is within the gene.

16. The method of claim 1, wherein the haplotype block is at least 50 kb distant from the gene.

17. The method of claim 1, wherein the haplotype block spans at least 10 kb.

18. The method of claim 1, wherein at least 80% of the haplotype patterns of one or more polymorphic sites in the haplotype block in the population are one of four or fewer distinct haplotype patterns.

19. The method of claim 1, wherein step (b) is repeated to determine which of the haplotype patterns at each of a plurality of haplotype blocks are associated with the differential relative allelic expression pattern.

20. The method of claim 19, wherein one haplotype block is within 50 kb of the gene, and a second haplotype block is at least 100 kb away from the gene on the same chromosome or is located on a different chromosome.

21. The method of claim 1, wherein the haplotype block is within 50 kb of the gene, and a first haplotype pattern of the haplotype block is associated with the differential relative allelic expression pattern, and the method further comprises repeating step (b) with a second haplotype block at least 100 kb from the gene or located on a different chromosome in a subset of the samples from individuals having the first haplotype pattern that is associated with the differential relative allelic expression pattern.

22. The method of claim 19, wherein the plurality of haplotype blocks comprises at least 25,000 blocks of polymorphic sites.

23. The method of claim 19, wherein the plurality of haplotype blocks comprises at least 100,000 blocks of polymorphic sites.

24. The method of claim 19, wherein the plurality of haplotype blocks comprises at least 200,000 blocks of polymorphic sites.

25. The method of claim 19, wherein the plurality of haplotype blocks comprises at least 500,000 blocks of polymorphic sites.

26. The method of claim 19, wherein the plurality of haplotype blocks comprises at least 1,000,000 blocks of polymorphic sites.

27. The method of claim19, wherein substantially all regions of the genome of the individuals are analyzed for association of haplotype patterns to the differential relative allelic expression pattern.

28. The method of claim 1, further comprising performing a clinical trial in which the identity of a drug a patient receives is determined by presence or absence in the patient of a haplotype pattern that is associated with the differential relative allelic expression pattern.

29. The method of claim 1, further comprising performing a clinical trial in which the dose of a drug a patient receives is determined by presence or absence in the patient of a haplotype pattern that is associated with the differential relative allelic expression pattern.

30. The method of claim 1, further comprising performing a clinical trial in which the dose and identity of a drug a patient receives is determined by presence or absence in the patient of a haplotype pattern that is associated with the differential relative allelic expression pattern.

31. The method of claim 1, further comprising performing a clinical trial in which a haplotype pattern that is associated with the differential relative allelic expression pattern is further analyzed to determine if the haplotype pattern is also associated with efficacy of a drug or treatment.

32. The method of claim 1, further comprising performing a clinical trial in which a haplotype pattern that is associated with the differential relative allelic expression pattern is further analyzed to determine if the haplotype pattern is also associated with an adverse response to a drug or treatment.

33. The method of claim 1, further comprising diagnosing a patient, wherein the presence or absence of a phenotypic trait is determined from presence or absence of a haplotype pattern that is associated with the differential relative allelic expression pattern.

34. The method of claim 33, wherein said phenotypic trait is one or more of a disease state, susceptibility to a disease, resistance to a disease, or response to a drug.

35. The method of claim 1, wherein the differential relative allelic expression pattern is determined by hybridizing mRNA or cDNA to a probe array.

36. The method of claim 1, wherein the differential relative allelic expression pattern is determined by performing a single base extension reaction using a primer having a 3′ end that hybridizes adjacent to a polymorphic site in the coding region of said gene.

37. The method of claim 1, wherein the differential relative allelic expression pattern is determined by sequencing RNA transcripts or nucleic acids derived therefrom.

38. The method of claim 1, wherein the differential relative allelic expression pattern is determined by allele-specific PCR amplification.

39. The method of claim 1, wherein the differential relative allelic expression pattern is determined by analyzing amino acid differences in proteins expressed from different alleles of the same gene.

40. The method of claim 1, further comprising determining whether expressed genes are partially or completely within or proximate to the haplotype block that contains one or more haplotype patterns associated with the differential relative allelic expression pattern.

41. The method of claim 40, wherein an expressed gene is located partially or completely within the haplotype block that contains one or more haplotype patterns associated with the differential relative allelic expression pattern and the method further comprises identifying an agent that alters the differential relative allelic expression pattern.

42. The method of claim 41, wherein the agent alters the differential relative allelic expression pattern by interacting with the protein encoded by the expressed gene.

43. The method of claim 41, wherein the agent alters the differential relative allelic expression pattern by interacting with the mRNA encoded by the expressed gene.

44. The method of claim 41, wherein the agent alters the differential relative allelic expression pattern by binding to an entity that interacts with the protein encoded by the expressed gene.

45. The method of claim 41, wherein the agent alters the differential relative allelic expression pattern by binding to an entity that interacts with the mRNA encoded by the expressed gene.

46. The method of claim 41, wherein the agent alters the differential relative allelic expression pattern by inhibiting or stimulating, either directly or indirectly, the transcription of the expressed gene.

47. The method of claim 41, wherein the agent alters the differential relative allelic expression pattern by inhibiting or stimulating, either directly or indirectly, the translation of the mRNA encoded by the expressed gene.

48. The method of claim 41, wherein the agent alters the differential relative allelic expression pattern by disrupting the activity of the protein encoded by the expressed gene.

49. The method of claim 41, wherein the agent alters the differential relative allelic expression pattern by disrupting the binding of the protein encoded by the expressed gene to DNA.

50. The method of claim 1, wherein said cells are isolated from a tissue selected from the list comprising blood, liver, brain, skin, kidney, breast, prostate, colon, muscle, nerve, lung,heart, stomach, connective tissue, bone marrow, and tumor tissue.

51. The method of claim 1, wherein step (b) identifies one or more haplotype patterns that are associated with the differential relative allelic expression patterns of the gene, and the one or more haplotype patterns are also associated with the differential relative allelic expression pattern of at least one other gene.

52. The method of claim 1, wherein a differential allelic expression pattern is determined for a plurality of genes, and step (b) is performed for each gene that exhibits a differential relative allelic expression pattern.

53. The method of claim 1, wherein step (b) identifies a plurality of haplotype patterns located in different haplotype blocks that are associated with the differential relative allelic expression pattern of the gene.

54. The method of claim 1, wherein step (b) identifies a plurality of haplotype patterns, at least two of which are located in the same haplotype block, and that are associated with the differential relative allelic expression pattern of the gene.

55. The method of claim 1, wherein step (b) identifies a plurality of haplotype patterns that cumulatively associate with the differential relative allelic expression pattern of the gene.

56. The method of claim 1, wherein step (b) identifies a plurality of haplotype patterns located in different haplotype blocks that are associated with differential relative allelic expression patterns of a plurality of different genes including the gene.

57. The method of claim 1, wherein step (b) identifies a plurality of haplotype patterns, at least two of which are located in the same haplotype block, and that are associated with differential relative allelic expression patterns of a plurality of different genes including the gene.

58. The method of claim 1, wherein step (b) identifies a plurality of haplotype patterns that cumulatively associate with differential relative allelic expression patterns of a plurality of different genes including the gene.

59. The method of claim 1 wherein no single polymorphic form in the haplotype block is solely responsible for causing the differential relative allelic expression patterns of the gene.

60. The method of claim 1, wherein the haplotype pattern is associated with differential gene expression and one of the polymorphic forms of the haplotype pattern is not directly involved in differential expresssion and the method further comprises using the polymorphic form as a marker to detect a second polymorphic form that is directly involved in the differential relative allelic expression pattern.

61. The method of claim 1, wherein a second gene is identified that overlaps at least in part with the haplotype block, wherein alteration of the expression level of the second gene or the function of its gene product alters the differential relative allelic expression pattern.

62. The method of claim 1, wherein the method identifies one or more haplotype patterns associated with the differential relative allelic expression pattern of the gene, and the method further comprises scanning one or more haplotype blocks containing the one or more haplotype patterns associated with the differential relative allelic expression pattern for the presence of expressed genes.

63. The method of claim 1, wherein step (b) identifies an associated haplotype pattern that is associated with the differential relative allelic expression pattern of said gene, and the method further comprises the step of performing an association analysis, wherein the test group is a subset of samples that exhibit the differential relative allelic expression pattern of the gene and have the associated haplotype pattern and the control group is a subset of samples that do not exhibit the differential relative allelic expression pattern of the gene and have the associated haplotype pattern, wherein a second associated haplotype pattern that is associated with the differential relative allelic expression pattern of the gene is identified.

64. The method of claim 1, wherein step (b) identifies an associated haplotype pattern that is associated with the differential relative allelic expression pattern of said gene, and the method further comprises the step of performing an association analysis, wherein a first group is a subset of samples that exhibits a first ratio of reference:alternate expression levels and has the associated haplotype pattern and a second group is a subset of samples that exhibits a second distinct ratio of reference: alternate expression levels and has the associated haplotype pattern, and further wherein a second associated haplotype pattern that is associated with the difference in magnitude of the first and second ratios is identified.

65. A method of characterizing a gene, comprising
(a) determining a differential relative allelic expression pattern of at least two alleles of said gene from samples containing diploid cells from a plurality of individuals of the same species, where said cells are heterozygous for said gene;
(b) determining whether the differential relative allelic expression pattern of the gene is associated with a polymorphic form at a polymorphic site outside the gene and regulatory regions that control the transcription thereof.


SEQUENCE LISTING (4 sequences)

SEQ ID NO: 1 - DNA, 13 bp, Artificial Sequence
  Figure 1 Individual W sequence
agattcgataacg

SEQ ID NO: 2 - DNA, 13 bp, Artificial
  Figure 1 Individual X sequence
agactacataacg

SEQ ID NO: 3 - DNA, 13 bp, Artificial
  Figure 1 Individual Y sequence
tatttcgataacg

SEQ ID NO: 4 - DNA, 13 bp, Artificial
  Figure 1 Individual Z sequence
tatctacaatcac